(19) United States
(12) Patent Application Publication
(10) Pub. No.: US 2026/0272425 A1
(43) Pub. Date: Sep. 17, 2026

(54) ADAPTIVELY WEIGHTED SPATIAL COMPOUNDING FOR ULTRASOUND IMAGE CONTRAST ENHANCEMENT

(71) Applicant: KONINKLIJKE PHILIPS N.V., EINDHOVEN (NL)

(72) Inventors: Iason Zacharias Apostolakis, Cambridge, MA (US); Jun Seob Shin, Winchester, MA (US); Faik Can Meral, Mansfield, MA (US); Jean-luc francois-marie Robert, Cambridge, MA (US); Jason Richard Yu, Arlington, MA (US); Changhong Hu, Bothell, WA (US); Thanasis Loupas, Kirkland, WA (US); Charles Tremblay-Darveau, Seattle, WA (US); James Robertson Jago, Bothell, WA (US)

(21) Appl. No.: 19/156,976

(22) PCT Filed: Feb. 7, 2024
(86) PCT No.: PCT/EP2024/052958

§ 371 Date: Aug. 15, 2025

Related U.S. Application Data
(60) Provisional application No. 63/446,183, filed on Feb. 16, 2023.

(51) Int. Cl. A61B 8/00; G01S 15/89

(52) U.S. Cl. CPC A61B 8/5253; A61B 8/5207; G01S 15/8995

(57) ABSTRACT

An ultrasound imaging system includes a processor that receives a plurality of images of a patient's internal anatomy obtained by an ultrasound imaging probe at a respective plurality of different angles. The processor computes an edge map for each respective image of the plurality of images. The processor uses the edge maps as weight maps to compute a weighted function of each corresponding pixel of each image in the plurality of images. The processor assembles a spatially compounded image based on values of the weighted functions of each corresponding pixel. The processor outputs the spatially compounded image on a display in communication with the processor.

### TECHNICAL FIELD

The subject matter described herein relates to systems, devices, and methods for enhancing the contrast of radiology images. This adaptively weighted spatial compounding system has particular but not exclusive utility for ultrasound imaging.

### BACKGROUND

Spatial compounding is a technique that reduces the appearance of speckles, shadows, and specular reflection discontinuities in ultrasound imaging. This is achieved by incoherently compounding multiple subframes that are obtained by insonifying a medium at different beam steering angles. Spatial compounding can increase the contrast-to-noise ratio (CNR), resulting in better image quality. Furthermore, spatial compounding helps reduce acoustic shadows created by suboptimal coupling of the probe, blockages, and/or anatomical structures that are highly attenuating. Spatial compounding also helps visualize tissue boundaries more clearly by making them more continuous. Because of such desirable effects, spatial compounding is often the default mode on many tissue-specific presets (TSPs) for ultrasound imaging systems.

Ultrasound image quality is highly dependent on the incidence angle of the ultrasound beam to the reflecting surface. Structures and surfaces where the incidence angle of the ultrasound beams is closest to normal produce strong echoes, leading to high contrast and enhanced tissue edge conspicuity. On the other hand, surfaces and structures that are tilted or off-axis with respect to the ultrasound beam may produce weak echoes, or even no echoes at all. Furthermore, with increased beam steering angle, structures and surfaces are further compromised by the increased presence of side lobes and grating lobe artifacts. In its current implementation, spatial compounding is a per-pixel averaging approach that assigns equal weights to echoes received from structures insonified with advantageous ultrasound beam incidence angles as well as to echoes received from the same structures but with oblique incidence angles. This indiscriminate combination of normal- and oblique-incidence-angle echoes can lead to reduced image contrast, and thus hinder tissue delineation because of the blurring of edges, ultimately lending certain regions of the image an oversmoothed appearance.

The information included in this Background section of the specification, including any references cited herein and any description or discussion thereof, is included for technical reference purposes only and is not to be regarded as subject matter by which the scope of the disclosure is to be bound.

### SUMMARY

Disclosed is an adaptively weighted spatial compounding system that combines ultrasound imaging subframes in a manner weighted to provide greater contrast to tissue boundaries and transitions. Adaptively weighted spatial compounding can also be referenced as adaptive spatial compounding. The present disclosure provides an adaptive version of spatial compounding that achieves enhanced image contrast, tissue delineation, border conspicuity, and imaging depth. The adaptive spatial compounding system includes a computationally efficient algorithm that improves image contrast by generating an edge map for each subframe, then normalizing the edge maps and using them as weights to produce a weighted average spatial compounding image. With this adaptive weighting system, the weight of the subframe with the most prominent tissue edge at each spatial location is increased, while the weights of subframes with less prominent tissue borders are reduced. This weighted averaging of the pixels in each subframe can lead to enhanced rendering of tissue borders and contrast-generating regions in the final, spatially compounded image.

The adaptive spatial compounding system disclosed herein has particular, but not exclusive, utility for image enhancement in ultrasound imaging streams (e.g., live, real time, or near real time, or post-processing). A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In an exemplary aspect, an ultrasound imaging system is provided. The ultrasound imaging system includes a processor configured for communication with a display. The processor is configured to receive a plurality of images of a patient's internal anatomy obtained by an ultrasound imaging probe at a respective plurality of different angles; for each respective image of the plurality of images, compute an edge map; using the edge maps as weight maps, compute a weighted function of each corresponding pixel of each image in the plurality of images; assemble a spatially compounded image based on values of the weighted functions of each corresponding pixel; and output the spatially compounded image on the display.

In some aspects, the ultrasound imaging system includes the ultrasound imaging probe. In some aspects, to receive the plurality of images, the processor is configured to control the ultrasound imaging probe to obtain the plurality of images at the respective plurality of different angles. In some aspects, the ultrasound image system includes a memory in communication with the processor, and, to receive the plurality of images, the processor is configured to retrieve the plurality of images from the memory. In some aspects, the processor is further configured to, for each respective image of the plurality of images, prior to computing the edge map: generate a corresponding despeckled image; generate a corresponding low-pass-filtered image; generate a corresponding subtraction image by subtracting the corresponding low-pass-filtered image from the corresponding despeckled image; and substitute the corresponding subtraction image for the respective image. In some aspects, the processor is further configured to, prior to outputting the spatially compounded image, perform gamma correction on the spatially compounded image. In some aspects, the processor is further configured to: for each respective image of the plurality of images: generate a corresponding despeckled image; generate a corresponding speckle image by subtracting the respective image from the despeckled image and replacing each pixel value with a corresponding absolute value of the pixel value, thus forming a plurality of speckle images; assemble a spatially compounded speckle image by computing a non-weighted function of each corresponding pixel of the plurality of speckle images; and add the spatially compounded speckle image to the spatially compounded image. In some aspects, a pixel representing an edge is enhanced in the spatially compounded image as compared with the plurality of images. In some aspects, a pixel representing a shadow is de-emphasized in the spatially compounded image as compared with the plurality of images. In some aspects, a pixel representing a side lobe artifact or grating lobe artifact is de-emphasized in the spatially compounded image as compared with the plurality of images. In some aspects, to use the edge maps as weight maps, the processor is configured to: pass the edge maps through a sigmoid function to achieve value scaling or thresholding; and normalize pixel values of the edge maps such that summing a given pixel across all of the edge maps yields a value of 1. In some aspects, the processor is further configured to align the images of the plurality of images with scan conversion. In some aspects, the weighted function comprises a weighted average.

In an exemplary aspect, an ultrasound imaging method is provided. The ultrasound imaging method includes receiving, with a processor, a plurality of images obtained by an ultrasound imaging probe at a respective plurality of different angles; computing, with the processor, an edge map for each respective image of the plurality of images; computing, with the processor, a weighted function of each corresponding pixel of each image in the plurality of images, using the edge maps as weight maps; assembling, with the processor, a spatially compounded image based on values of the weighted functions of each corresponding pixel; and outputting, with the processor, the spatially compounded image on a display in communication with the processor.

In an exemplary aspect, a non-transitory computer-readable storage medium is provided. The non-transitory computer-readable storage medium has program code recorded thereon. The program code comprises instructions executable by a processor of an ultrasound imaging system to cause the ultrasound imaging system to: receive a plurality of images of a patient's internal anatomy obtained by an ultrasound imaging probe at a respective plurality of different angles; for each respective image of the plurality of images, compute an edge map; using the edge maps as weight maps, compute a weighted function of each corresponding pixel of each image in the plurality of images; assemble a spatially compounded image based on values of the weighted functions of each corresponding pixel; and output the spatially compounded image on a display in communication with the processor.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the adaptive spatial compounding system, as defined in the claims, is provided in the following written description of various aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1 is a diagrammatic schematic view of an ultrasound imaging system, according to aspects of the present disclosure.

FIG. 2 is a schematic diagram of a processor circuit, according to aspects of the present disclosure. is a schematic diagram of a processor circuit, according to aspects of the present disclosure.

FIG. 3 is a schematic, diagrammatic illustration of a spatial compounding process, in accordance with at least one aspect of the present disclosure.

FIG. 4 is an example of a spatial compounding process, in accordance with at least one aspect of the present disclosure.

FIG. 5A is an example subframe for a spatial compounding process, in accordance with at least one aspect of the present disclosure.

FIG. 5B is an example spatially compounded image, in accordance with at least one aspect of the present disclosure.

FIG. 6 is a schematic, diagrammatic representation of a circular object being imaged at two different scan angles, in accordance with at least one aspect of the present disclosure.

FIG. 7 is a simplified example of a spatial compounding process, in accordance with at least one aspect of the present disclosure.

FIG. 8 is a simplified example of an adaptive spatial compounding process, in accordance with at least one aspect of the present disclosure.

FIG. 9 shows a flow diagram of an example adaptive spatial compounding method, in accordance with at least one aspect of the present disclosure.

FIG. 10 is a simplified example of an adaptive spatial compounding process, in accordance with at least one aspect of the present disclosure.

FIG. 11 shows an example adaptive spatial compounding weight calculation method, in a hybrid flow diagram/block diagram form, in accordance with at least one aspect of the present disclosure.

FIG. 12A shows an example spatial compounding image produced by existing spatial compounding techniques from the subframes of FIG. 10, in accordance with at least one aspect of the present disclosure.

FIG. 12B shows an example adaptive spatial compounding image produced by the adaptive spatial compounding system, from the subframes FIG. 10, in accordance with at least one aspect of the present disclosure.

FIG. 13A shows an example spatial compounding image produced by existing spatial compounding techniques from subframes of FIG. 4, in accordance with at least one aspect of the present disclosure.

FIG. 13B shows an example adaptive spatial compounding image produced by the adaptive spatial compounding system, in accordance with at least one aspect of the present disclosure.

FIG. 14 is an illustration of the effect of gamma correction on an adaptive spatial compounding system image, in accordance with at least one aspect of the present disclosure.

FIG. 15 shows a flow diagram of an example 2-scale adaptive spatial compounding method, in accordance with at least one aspect of the present disclosure.

FIG. 16 shows an example 2-scale adaptive spatial compounding weight calculation method, in a hybrid flow diagram/block diagram form, in accordance with at least one aspect of the present disclosure.

FIG. 17A shows an example adaptive spatial compounding image produced by the adaptive spatial compounding system, from the subframes FIG. 10, in accordance with at least one aspect of the present disclosure.

FIG. 17B shows an example 2-scale adaptive spatial compounding image produced by the adaptive 2-scale spatial compounding system of FIGS. 15 and 16, from the subframes of FIG. 10, in accordance with at least one aspect of the present disclosure.

### DETAILED DESCRIPTION

In accordance with at least one aspect of the present disclosure, an adaptive spatial compounding system is provided which combines ultrasound imaging subframes in a manner weighted to provide greater contrast to tissue boundaries and transitions.

Spatial compounding is a technique that improves ultrasound image quality by incoherently compounding multiple subframes obtained at different beam angles. However, the indiscriminate combination of echoes obtained at normal and oblique incidence angles leads to reduced image contrast. More specifically, ultrasound image quality is highly dependent on the incidence angle of the ultrasound beam to the reflecting surface, with structures and surfaces that are perpendicular to the beam returning stronger echoes than structures and surfaces oriented obliquely to the beam. Increasing the beam steering angle can increase the presence of side lobes and grating lobe artifacts, thus making the echoes from oblique surfaces even harder to distinguish. Current implementations of spatial compounding do not consider local contrast or tissue edge conspicuity in each angled subframe. More specifically, current forms of spatial compounding use a per-pixel averaging approach that assigns equal weights to echoes received from structures at advantageous (perpendicular) incidence angles and those received from unfavorable (oblique) incidence angles. This indiscriminate combination of normal and oblique incidence angle echoes can average faint echoes with strong ones, which reduces the image contrast, and thus hinders tissue delineation because of the blurring of edges. Certain regions of the image can thus have an oversmoothed appearance.

To address this issue, the present disclosure provides an adaptive version of spatial compounding that achieves enhanced image contrast, tissue delineation, and border conspicuity. The adaptive spatial compounding approach includes an algorithm of low complexity that improves image contrast by obtaining smooth, per-pixel estimates of tissue edges for each subframe, subsequently normalizing them and then using them as weights to produce a weighted average spatial compounding image. With this adaptive weighting system, the blending weight of the subframe with the most prominent tissue edge at each spatial location is boosted, while the subframes with less prominent tissue borders are de-emphasized. This weighted averaging of the pixels in each subframe can therefore lead to enhanced rendering of tissue borders and contrast-generating regions when the final, spatially compounded image is assembled from the averaged pixels. As a side effect, this process also reduces the appearance of shadows, side lobes, and grating lobe artifacts because the “edge absences” produced by such artifacts will not be identical between different subframes captured at different angles. Combining the non-identical subframes effectively cancels, minimizes, and/or otherwise de-emphasizes the shadows, side lobes, and grating lobe artifacts in the spatially compounded image. For example, a pixel that represents or forms a portion of such an image artifact in the spatially compounded image and/or one subframe is reduced in appearance relative to the same pixel in a different subframe. At the same time as the shadows, side lobes, and grating lobe artifacts are de-emphasized, the adaptive weighting increase the conspicuity or otherwise emphasizes the edges in the spatially compounded image.

In one aspect of the adaptive spatial compounding system, edge-based weight maps are computed for each spatial compounding subframe according to an estimate of the image gradient, or other edge detection method, followed by a normalization step. Consequently, the computed edge-based weight maps prioritize the view that produces the most prominent tissue edge at each pixel. Thus, in image regions with tissue edges, the subframe that produces the most prominent edge is given a higher weight value compared to the other subframes. Meanwhile, in homogeneous tissue areas or in areas producing little to no echoes, all edge maps may yield similarly low gradient/edge values and thus the weighting scheme tends to assign relatively similar values to all subframes, reverting to the default spatial compounding image appearance.

Previous work in advanced spatial compounding has included comparing various simple incoherent spatial compounding operations (e.g., averaging, median estimation, maximum estimation etc.). Some more complex algorithms have been proposed that attempt to enhance tissue edges (while in some cases suppressing artifacts) via image transformation or pyramid decomposition. In one example, the authors identify prominent surfaces in the angled image subframes, and subsequently perform beam-surface angle detection in order to calculate a weighted average of the images. In that example, the local edge detector is implemented as a thresholding operation on the directional image gradient along the ultrasound beam direction. Such a simplistic approach can be inaccurate in the presence of complex morphology, and can also be vulnerable to certain ultrasound artifacts and to increased speckle noise.

In another example, the authors obtain the monogenic signal representation of the image (an isotropic extension of analytic signal for N-dimensional signals) and compute a phase congruency feature across multiple spatial scales to detect salient image features (prominent lines and edges) as well as utilizing the phase of the monogenic signal to estimate the beam-tissue incidence angle. In still another example, the first steps of the methodology involve decomposing the image into Laplacian and Gaussian pyramids. Subsequently, a confidence metric derived from each subframe image is also decomposed into Gaussian pyramids. A decision is then made for each pixel at each spatial scale according to the confidence metric, regarding whether it corresponds to real tissue or an ultrasound artifact. If the pixel is classified as tissue, then the pixel from the angled subframe with the highest contrast is selected. Otherwise, the pixel with the highest confidence metric is selected. While this approach ensures that contrast is not enhanced for ultrasound artifacts, the classification criteria are not sufficiently robust, resulting in certain ultrasound artifacts getting enhanced and certain tissue edges getting no contrast improvement.

Three common problems are found across currently available advanced spatial compounding methodologies. First, advanced spatial compounding approaches that directly use the image gradient as a way of detecting image lines and borders may be very sensitive to increased speckle variance and ultrasound artifacts. Consequently, a smoother approach of identifying image lines is necessary. Second, several advanced methodologies consider the nominal steering angle of the angled spatial compounding subframe, essentially making the simplifying assumption that the ultrasound transmissions form a single plane wave steered according to that nominal steering angle. This assumption, while partially correct, ignores the fact that the steered, focused transmissions can be decomposed into multiple plane waves, insonifying the field of view from a plurality of directions, especially for features closer to the focal point. This simplification is further accentuated by imaging modalities that combine neighboring beams to achieve an extended depth of field. Consequently, limited image quality improvements may be seen in algorithms that rely on weighting the subframes according to only the nominal steering angles, without any data-driven contrast estimation. Third, computationally complex methodologies may not be suitable for real-time imaging on the computing hardware available in the ultrasound console.

Additionally, spatial compounding algorithms have been devised to enhance needle visualization in ultrasound-guided needle insertion operations. It should be noted, however, that several of these algorithms use application-specific prior knowledge e.g., using directional filters to enhance contrast along certain beam directions that are expected to yield improved needle visualization due to the expected angle of needle insertion.

The present disclosure overcomes these difficulties by providing robust, repeatable, computationally inexpensive spatial compounding algorithms that can effectively enhance tissue features while reducing the appearance of image artifacts. In one exemplary aspect, the adaptive spatial compounding system includes an algorithm of low complexity that aims to improve spatial compounding image contrast by obtaining smooth estimates of tissue edges for each spatial compounding subframe image, subsequently normalizing them and then using them as weights to produce a weighted average image, or an image produced by a linear or nonlinear weighted function other than averaging. Note that the weighted combination of the images could also involve transforming the image and weight data into some other domain (e.g., spatial frequency domain via 2-D Fourier transform), followed by a weighted combination of the data and an inverse transform back into the image domain. Unlike previous attempts, the present disclosure provides clear strategies for calculating the per-pixel weights of each subframe image in order to enhance image quality.

The present disclosure provides an approach for calculating the averaging weights to achieve enhanced image sharpness, tissue conspicuity, artifact reduction, and improved imaging depth. The present disclosure can also be framed as a multi-steering-angle ultrasound image sharpness enhancement technique, e.g., a unique end-to-end algorithm that achieves specific enhanced image quality characteristics. It improves over other advanced spatial compounding techniques by providing a complete methodology including weight calculation as well as the anticipated effects on the ultrasound image (sharpness, tissue conspicuity, etc.). In other aspects, the adaptive spatial compounding system can serve as an extension (e.g., a software update) to spatial compounding systems already developed and deployed.

The present disclosure aids substantially in ultrasound image enhancement, by improving spatial compounding outcomes. The adaptive spatial compounding system effectively decreases the occurrence of speckle, shadowing, and other image artifacts, while enhancing the appearance of edges and tissue boundaries, and improving imaging depth. Implemented on a processor in communication with an ultrasound probe, the adaptive spatial compounding system disclosed herein provides practical improvements in medical procedures involving live ultrasound imaging. This improved real-time image enhancement transforms a spatial compounding process that reduces image contrast into one that enhances the appearance of edges and tissue boundaries, without the normally routine need for a clinician to gather images over multiple seconds in order to gain understanding of the patient's internal anatomy. This unconventional approach improves the functioning of the ultrasound imaging system, by providing clearer, more anatomically accurate real-time images to the clinician.

The adaptive spatial compounding system may be implemented as a process whose outputs are viewable on a display, and operated by a control process executing on a processor that accepts user inputs from a keyboard, mouse, or touchscreen interface, and that is in real-time communication with an ultrasound probe and a memory. In that regard, the control process performs certain specific operations in response to different inputs or selections made at different times. Certain structures, functions, and operations of the processor, display, sensors, and user input systems are known in the art, while others are recited herein to enable novel features or aspects of the present disclosure with particularity.

These descriptions are provided for exemplary purposes only, and should not be considered to limit the scope of the adaptive spatial compounding system. Certain features may be added, removed, or modified without departing from the spirit of the claimed subject matter.

For the purposes of promoting an understanding of the principles of the present disclosure, reference will now be made to the aspects illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one aspect may be combined with the features, components, and/or steps described with respect to other aspects of the present disclosure. For the sake of brevity, however, the numerous iterations of these combinations will not be described separately.

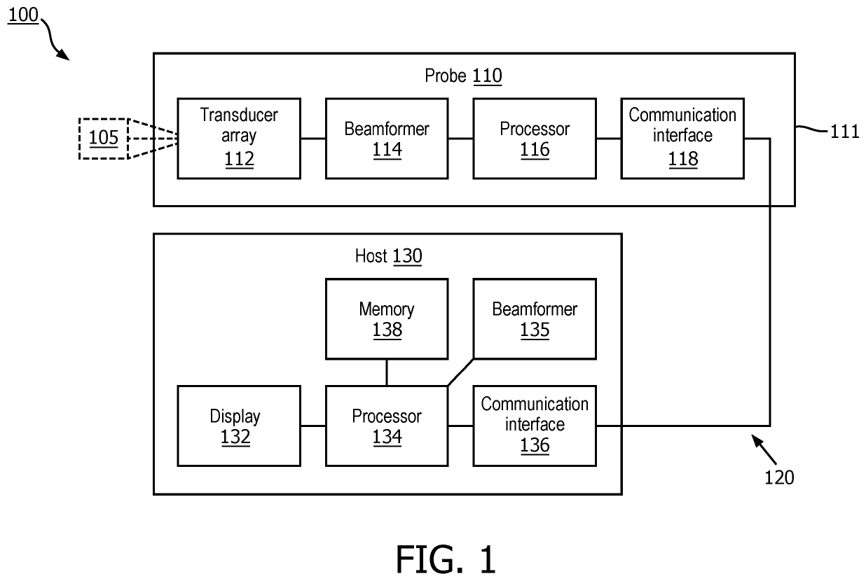

FIG. 1 is a diagrammatic schematic view of an ultrasound imaging system, according to aspects of the present disclosure. The system 100 is used for scanning an area or volume of a patient's body. The system 100 includes an ultrasound imaging probe 110 in communication with a host 130 over a communication interface or link 120. The probe 110 may include a transducer array 112, a beamformer 114, a processor 116, and a communication interface 118. The host 130 may include a display 132, a processor circuit 134, a communication interface 136, and a memory 138 storing patient information.

In some aspects, the probe 110 is an external ultrasound imaging device including a housing 111 configured for handheld operation by a user. The transducer array 112 can be configured to obtain ultrasound data while the user grasps the housing 111 of the probe 110 such that the transducer array 112 is positioned adjacent to or in contact with a patient's skin. The probe 110 is configured to obtain ultrasound data of anatomy within the patient's body while the probe 110 is positioned outside of the patient's body. In some aspects, the probe 110 can be a patch-based external ultrasound probe.

In other aspects, the probe 110 can be an internal ultrasound imaging device and may comprise a housing 111 configured to be positioned within a lumen of a patient's body, including the patient's coronary vasculature, peripheral vasculature, esophagus, heart chamber, or other body lumen or body cavity. In some aspects, the probe 110 may be an intravascular ultrasound (IVUS) imaging catheter or an intracardiac echocardiography (ICE) catheter. In other aspects, probe 110 may be a transesophageal echocardiography (TEE) probe. Probe 110 may be of any suitable form for any suitable ultrasound imaging application including both external and internal ultrasound imaging.

In some aspects, aspects of the present disclosure can be implemented with medical images of patients obtained using any suitable medical imaging device and/or modality. Examples of medical images and medical imaging devices include x-ray images (angiographic images, fluoroscopic images, images with or without contrast) obtained by an x-ray imaging device, computed tomography (CT) images obtained by a CT imaging device, positron emission tomography-computed tomography (PET-CT) images obtained by a PET-CT imaging device, magnetic resonance images (MRI) obtained by an MRI device, single-photon emission computed tomography (SPECT) images obtained by a SPECT imaging device, optical coherence tomography (OCT) images obtained by an OCT imaging device, and intravascular photoacoustic (IVPA) images obtained by an IVPA imaging device. The medical imaging device can obtain the medical images while positioned outside the patient body, spaced from the patient body, adjacent to the patient body, in contact with the patient body, and/or inside the patient body.

For an ultrasound imaging device, the transducer array 112 emits ultrasound signals towards an anatomical object 105 of a patient and receives echo signals reflected from the object 105 back to the transducer array 112. The transducer array 112 can include any suitable number of acoustic elements, including one or more acoustic elements and/or a plurality of acoustic elements. In some instances, the transducer array 112 includes a single acoustic element. In some instances, the transducer array 112 may include an array of acoustic elements with any number of acoustic elements in any suitable configuration. For example, the transducer array 112 can include between 1 acoustic element and 10000 acoustic elements, including values such as 2 acoustic elements, 4 acoustic elements, 36 acoustic elements, 64 acoustic elements, 128 acoustic elements, 500 acoustic elements, 812 acoustic elements, 1000 acoustic elements, 1920 acoustic elements, 3000 acoustic elements, 8000 acoustic elements, and/or other values both larger and smaller. In some instances, the transducer array 112 may include an array of acoustic elements with any number of acoustic elements in any suitable configuration, such as a linear array, a planar array, a curved array, a curvilinear array, a circumferential array, an annular array, a phased array, a matrix array, a one-dimensional (1D) array, a 1.x dimensional array (e.g., a 1.5D array), or a two-dimensional (2D) array. The array of acoustic elements (e.g., one or more rows, one or more columns, and/or one or more orientations) can be uniformly or independently controlled and activated. The transducer array 112 can be configured to obtain one-dimensional, two-dimensional, and/or three-dimensional images of a patient's anatomy. In some aspects, the transducer array 112 may include a piezoelectric micromachined ultrasound transducer (PMUT), capacitive micromachined ultrasonic transducer (CMUT), single crystal, lead zirconate titanate (PZT), PZT composite, other suitable transducer types, and/or combinations thereof.

The object 105 may include any anatomy or anatomical feature, such as a diaphragm, blood vessels, nerve fibers, airways, mitral leaflets, cardiac structure, abdominal tissue structure, appendix, large intestine (or colon), small intestine, kidney, liver, and/or any other anatomy of a patient. In some aspects, the object 105 may include at least a portion of a patient's large intestine, small intestine, cecum pouch, appendix, terminal ileum, liver, epigastrium, and/or psoas muscle. The present disclosure can be implemented in the context of any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood vessels, blood, chambers or other parts of the heart, abdominal organs, and/or other systems of the body. In some aspects, the object 105 may include malignancies such as tumors, cysts, lesions, hemorrhages, or blood pools within any part of human anatomy. The anatomy may be a blood vessel, as an artery or a vein of a patient's vascular system, including cardiac vasculature, peripheral vasculature, neural vasculature, renal vasculature, and/or any other suitable lumen inside the body. In addition to natural structures, the present disclosure can be implemented in the context of man-made structures such as, but without limitation, heart valves, stents, shunts, filters, implants and other devices.

The beamformer 114 is coupled to the transducer array 112. The beamformer 114 controls the transducer array 112, for example, for transmission of the ultrasound signals and reception of the ultrasound echo signals. In some aspects, the beamformer 114 may apply a time-delay to signals sent to individual acoustic transducers within an array in the transducer array 112 such that an acoustic signal is steered in any suitable direction propagating away from the probe 110. The beamformer 114 may further provide image signals to the processor 116 based on the response of the received ultrasound echo signals. The beamformer 114 may include multiple stages of beamforming. The beamforming can reduce the number of signal lines for coupling to the processor 116. In some aspects, the transducer array 112 in combination with the beamformer 114 may be referred to as an ultrasound imaging component.

The processor 116 is coupled to the beamformer 114. The processor 116 may also be described as a processor circuit, which can include other components in communication with the processor 116, such as a memory, beamformer 114, communication interface 118, and/or other suitable components. The processor 116 may include a central processing unit (CPU), a graphical processing unit (GPU), a digital signal processor (DSP), an application specific integrated circuit (ASIC), a controller, a field programmable gate array (FPGA) device, another hardware device, a firmware device, or any combination thereof configured to perform the operations described herein. The processor 116 may also be implemented as a combination of computing devices, e.g., a combination of a DSP and a microprocessor, a plurality of microprocessors, one or more microprocessors in conjunction with a DSP core, or any other such configuration. The processor 116 is configured to process the beamformed image signals. For example, the processor 116 may perform filtering and/or quadrature demodulation to condition the image signals. The processor 116 and/or 134 can be configured to control the array 112 to obtain ultrasound data associated with the object 105.

The communication interface 118 is coupled to the processor 116. The communication interface 118 may include one or more transmitters, one or more receivers, one or more transceivers, and/or circuitry for transmitting and/or receiving communication signals. The communication interface 118 can include hardware components and/or software components implementing a particular communication protocol suitable for transporting signals over the communication link 120 to the host 130. The communication interface 118 can be referred to as a communication device or a communication interface module.

The communication link 120 may be any suitable communication link. For example, the communication link 120 may be a wired link, such as a universal serial bus (USB) link or an Ethernet link. Alternatively, the communication link 120 may be a wireless link, such as an ultra-wideband (UWB) link, an Institute of Electrical and Electronics Engineers (IEEE) 802.11 WiFi link, or a Bluetooth link.

At the host 130, the communication interface 136 may receive the image signals. The communication interface 136 may be substantially similar to the communication interface 118. The host 130 may be any suitable computing and display device, such as a workstation, a personal computer (PC), a laptop, a tablet, or a mobile phone.

The processor 134 is coupled to the communication interface 136. The processor 134 may also be described as a processor circuit, which can include other components in communication with the processor 134, such as the memory 138, the communication interface 136, and/or other suitable components. The processor 134 may be implemented as a combination of software components and hardware components. The processor 134 may include a central processing unit (CPU), a graphics processing unit (GPU), a digital signal processor (DSP), an application-specific integrated circuit (ASIC), a controller, an FPGA device, another hardware device, a firmware device, or any combination thereof. The processor 134 can be configured to generate image data from the image signals received from the probe 110. The processor 134 can apply advanced signal processing and/or image processing techniques to the image signals. An example of image processing includes conducting a pixel level analysis to evaluate whether there is a change in the color of a pixel, which may correspond to an edge of an object (e.g., the edge of an anatomical feature). In some aspects, the processor 134 can form a three-dimensional (3D) volume image from the image data. In some aspects, the processor 134 can perform real-time processing on the image data to provide a streaming video of ultrasound images of the object 105.

The memory 138 is coupled to the processor 134. The memory 138 may be any suitable storage device, or a combination of different types of memory. The memory 138 can be configured to store patient information, measurements, data, or files relating to a patient's medical history, history of procedures performed, anatomical or biological features, characteristics, or medical conditions associated with a patient, computer readable instructions, such as code, software, or other application, as well as any other suitable information or data. The memory 138 may be located within the host 130. Patient information may include measurements, data, files, other forms of medical history, such as but not limited to ultrasound images, ultrasound videos, and/or any imaging information relating to the patient's anatomy.

Any or all of the previously mentioned computer readable media, such as patient information, code, software, or other applications, or any other suitable information or data may also be stored the memory 140. The memory 140 may serve a substantially similar purpose to the memory 138 but may not be located within the host 130. For example, in some aspects, the memory may be a cloud-based server, an external storage device, or any other device for memory storage. The host 130 may be in communication with the memory 140 by any suitable means as described. The host 130 may be in communication with the memory 140 continuously or they may be in communication intermittently upon the request of the host 130 or a user of the ultrasound system 100.

The host 130 may be in communication with the memory 140 via any suitable communication method. For example, the host 130 may be in communication with the memory 140 via a wired link, such as a USB link or an Ethernet link. Alternatively, the host 130 may be in communication with the memory 140 via a wireless link, such as an UWB link, an IEEE 802.11 WiFi link, or a Bluetooth link.

The display 132 is coupled to the processor circuit 134. The display 132 may be a monitor or any suitable display. The display 132 is configured to display the ultrasound images, image videos, and/or any imaging information of the object 105. In some aspects, the host 130 may include a beamformer circuit 135. For example, the system 100 may include a partial beamformer or microbeamformer 114 in the probe 110 (performing one or a plurality of initial stages of beamforming), and a main beamformer 135 in the console host 130 (performing one or a plurality of subsequent stages of beamforming). In some aspects, the system 100 may include a beamformer only in the probe 110 or only in the host 130, or other arrangements depending on the implementation.

The system 100 may be used to assist a sonographer in performing an ultrasound scan. The scan may be performed in a at a point-of-care setting. In some instances, the host 130 is a console or movable cart. In some instances, the host 130 may be a mobile device, such as a tablet, a mobile phone, or portable computer.

Before continuing, it should be noted that the examples described above are provided for purposes of illustration, and are not intended to be limiting. Other devices and/or device configurations may be utilized to carry out the operations described herein.

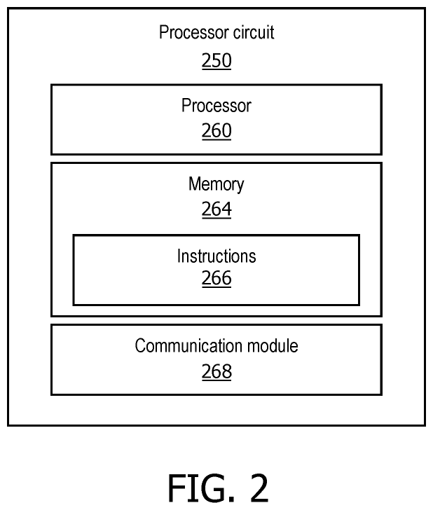

FIG. 2 is a schematic diagram of a processor circuit 250, according to exemplary aspects of the present disclosure. The processor circuit 250 may be implemented in the system 100, or other devices or workstations (e.g., third-party workstations, network routers, etc.), or on a cloud processor or other remote processing unit, as necessary to implement the method. As shown, the processor circuit 250 may include a processor 260, a memory 264, and a communication module 268. These elements may be in direct or indirect communication with each other, for example via one or more buses.

The processor 260 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 260 may also comprise another hardware device, a firmware device, or any combination thereof configured to perform the operations described herein. The processor 260 may also be implemented as a combination of computing devices, e.g., a combination of a DSP and a microprocessor, a plurality of microprocessors, one or more microprocessors in conjunction with a DSP core, or any other such configuration.

The memory 264 may include a cache memory (e.g., a cache memory of the processor 260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an aspect, the memory 264 includes a non-transitory computer-readable medium. The memory 264 may store instructions 266. The instructions 266 may include instructions that, when executed by the processor 260, cause the processor 260 to perform the operations described herein. Instructions 266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 268 can include any electronic circuitry and/or logic circuitry to facilitate direct or indirect communication of data between the processor circuit 250, and other processors or devices. In that regard, the communication module 268 can be an input/output (I/O) device. In some instances, the communication module 268 facilitates direct or indirect communication between various elements of the processor circuit 250 and/or the system 100. The communication module 268 may communicate within the processor circuit 250 through numerous methods or protocols. Serial communication protocols may include but are not limited to United States Serial Protocol Interface (US SPI), Inter-Integrated Circuit (I2C), Recommended Standard 232 (RS-232), RS-485, Controller Area Network (CAN), Ethernet, Aeronautical Radio, Incorporated 429 (ARINC 429), MODBUS, Military Standard 1553 (MIL-STD-1553), or any other suitable method or protocol. Parallel protocols include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, preset sharing between the processor and central server, or readings from the ultrasound probe) may be accomplished using any suitable wireless or wired communication technology, such as a cable interface such as a universal serial bus (USB), micro USB, Lightning, or FireWire interface, Bluetooth, Wi-Fi, ZigBee, Li-Fi, or cellular data connections such as 2G/GSM (global system for mobiles), 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

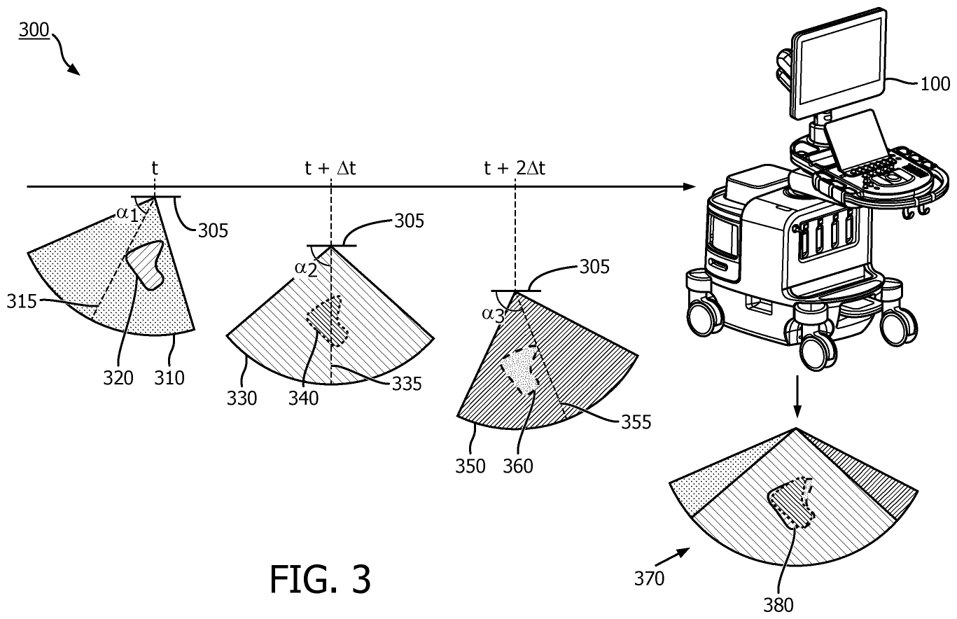

FIG. 3 is a schematic, diagrammatic illustration of a spatial compounding process 300, in accordance with at least one aspect of the present disclosure. One example spatial compounding process is the SonoCT feature included in various Philips ultrasound systems. Spatial compounding is described for example in U.S. Pat. No. 6,210,328, which is hereby incorporated by reference as though fully set forth herein. Spatial compounding is an imaging technique in which a number of ultrasound images of a given target that have been obtained from multiple vantage points or angles (look directions) are combined into a single compounded image by combining the data received from each point in the compound image target which has been received from each angle.

Other examples of spatial compounding may be found in U.S. Pat. Nos. 4,649,927; 4,319,489; and 4,159,462, which are hereby incorporated by reference as though fully set forth herein. Real time spatial compound imaging is performed by rapidly acquiring a series of partially overlapping component image frames from substantially independent spatial directions, utilizing an array transducer to implement electronic beam steering and/or electronic translation of the component frames, or by having a clinician physically move or reorient the ultrasound probe. The component frames are combined into a compound image by summation, averaging, peak detection, or other combinational means. The acquisition sequence and formation of compound images are repeated continuously at a rate limited by the acquisition frame rate, that is, the time required to acquire the full complement of scanlines over the selected width and depth of imaging. The compounded image typically shows lower speckle and better specular reflector delineation than conventional ultrasound images from a single viewpoint.

In the example shown in FIG. 3, at a time t, the ultrasound system 100 captures a first ultrasound image 310 at a beam angle of α1 (e.g., an angle between a transducer face 305 and a central axis 315 of the first ultrasound image 310). The ultrasound image 310 includes a first view 320 of an anatomical feature. At a second time t+Δt, the ultrasound system 100 captures a second ultrasound image 330, with a second view 340 of the anatomical feature, at a beam angle of α2 (e.g., an angle between the transducer face 305 and a central axis 335 of the second ultrasound image 330). At a third time t+2Δt, the ultrasound system 100 captures a third ultrasound image 350, with a third view 360 of the anatomical feature, at a beam angle of α3 (e.g., an angle between the transducer face 305 and a central axis 355 of the third ultrasound image 350). In the illustrated example, the central axis 335 of the second ultrasound image 330 is perpendicular to the transducer face 305. For example, the beam angle α2 is nominally 90°, the beam angle α1 is nominally less than 90° (e.g., between 0° and) 89°, and the beam angle «3 is nominally greater than 90° (e.g., between 91° and) 180°. In some instances, the central axis 335 being perpendicular to the transducer face 305 can be considered as a default or a reference. For example, the beam angle α2 can be described as 0° (e.g., not angled relative to the default/reference). The beam angle α2 is less than 0° (e.g., angled in one direction relative to the default/reference, to the left in FIG. 3), such as between −1° and −90°, and the beam angle «3 is greater than 0° (e.g., angled in the opposite direction relative to the default/reference, to the right in FIG. 3), such as between 1° and 90°, or vice versa.

It is noted that in some cases (e.g., with a matrix transducer array or other 3D imaging system), it may be possible to capture multiple angles simultaneously. In some instances, receive beamforming can be performed in order to generate multiple angles on the receive side from a same transmit event. For example, there can be one transmission of sound, and then beamforming circuitry can delay and sum simultaneously to form angled beams. Each ultrasound image 310, 330, 350 includes features that may not be visible in the other ultrasound images, and imaging artifacts (e.g., speckle, shadows) that may not be present in the other images, or may be present to a different degree or have a different appearance. Thus, each image 310, 330, 350 contains information that may not be present in the other images.

The ultrasound system 100 (e.g., a processor of the ultrasound system 100) therefore averages or otherwise combines the three images 310, 330, 350 to produce a combined image 370, which includes a combined view 380 of the anatomical feature. The combined view 380 of the anatomical feature may include all of the features visible in any of the views 320, 340, 360, and may thus provide greater insight to a clinician operating the ultrasound system 100. Depending on the implementation, this image combining process, image averaging process, or spatial compounding process may occur on an ongoing basis, e.g. with a moving average of the three most recent image frames. The process may also use more or fewer images, e.g., the most recent two images, the most recent five images, etc., although smaller numbers of images may provide less image enhancement, and larger numbers of images may both increase the computational burden and create a blurring effect in the presence of motion.

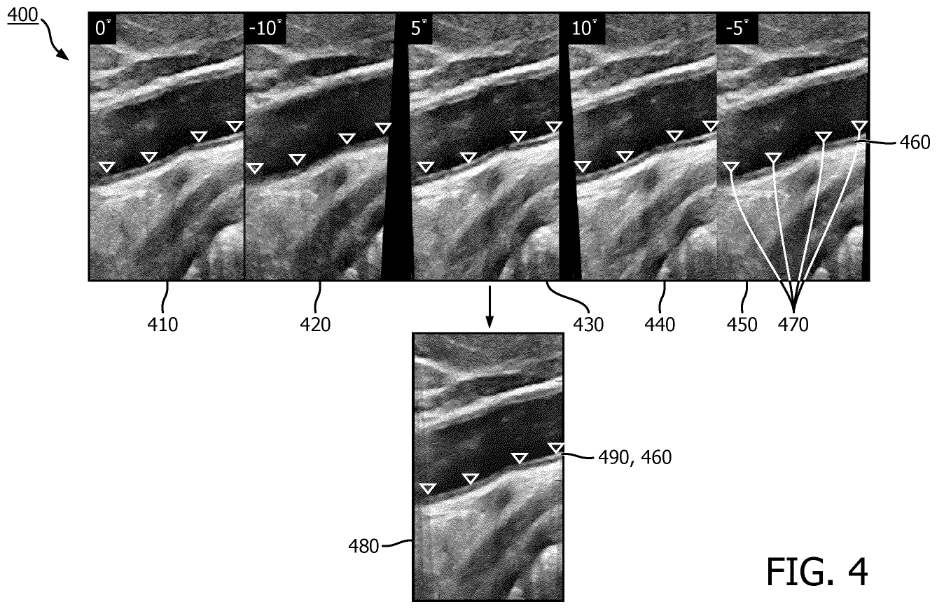

FIG. 4 is an example of a spatial compounding process 400, in accordance with at least one aspect of the present disclosure. In the example shown in FIG. 4, five subframe images 410, 420, 430, 440, and 450 are captured at respective beam angles of 0°,−10°, 5°, 10°, and −5°, although other numbers of frames, both greater and smaller, may be used instead or in addition. Each subframe 410, 420, 430, 440, and 450 includes a partial view of an anatomical feature 460 (in this case, a vessel wall), indicated by markers 470. The subframes can also be referred to as images or image frames.

These subframes 410, 420, 430, 440, and 450 are positionally and rotationally registered to one another in a scan conversion step, and then combined (e.g., averaged) to assemble a spatially compounded image 480 that includes an averaged view 490 of the anatomical feature 460, which includes contributions from all the subframes views of the anatomical feature 460, and may thus provide a more complete representation of the anatomical feature 460. Scan conversion can include transforming the image data from one coordinate system (e.g., used for obtaining and/or processing the image data), such as polar coordinates, to a different coordinate system (e.g., used for display), such as cartesian coordinates. For example, after scan conversion, the subframes 410, 420, 430, 440, and 450 as a whole can have the same image size in cartesian coordinates (e.g., a quantity of pixels in x dimension, a quantity of pixels in y dimension). The scan conversion results in the subframes 410, 420, 430, 440, and 450 occupying different portions of the same size image (because the subframes 410, 420, 430, 440, and 450 are captured at different beam angles). This effectively positionally and rotationally registers the subframes 410, 420, 430, 440, and 450 because the same (x, y) coordinate represents the same location in the patient body across all of the subframes 410, 420, 430, 440, and 450. It is understood that any suitable coordinate systems can be used.

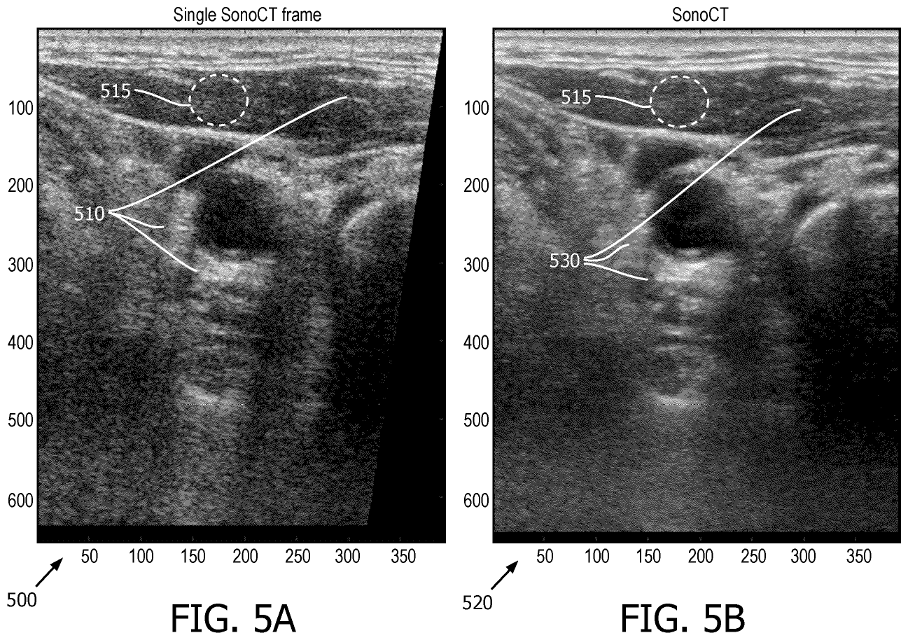

FIG. 5A is an example subframe 500 for a spatial compounding process, in accordance with at least one aspect of the present disclosure. The subframe includes a number of shadows 510 where the ultrasound beam has returned no echoes (e.g., because of interfering structures within the patient, or for other reasons). These shadows 510 are image artifacts that do not reflect real anatomy, and they may in fact obscure real anatomy in the locations where the shadows 510 occur.

FIG. 5B is an example spatially compounded image 520, in accordance with at least one aspect of the present disclosure. Since the spatially compounded image is an average or similar combination of ultrasound images captured at slightly different angles, it contains information that may be hidden by the shadows 510 in FIG. 5A. Thus, the spatially compounded image 520 includes attenuated shadows 530 that may, in some areas, be difficult to perceive at all. The spatially compounded image also has a reduced appearance of speckles 515.

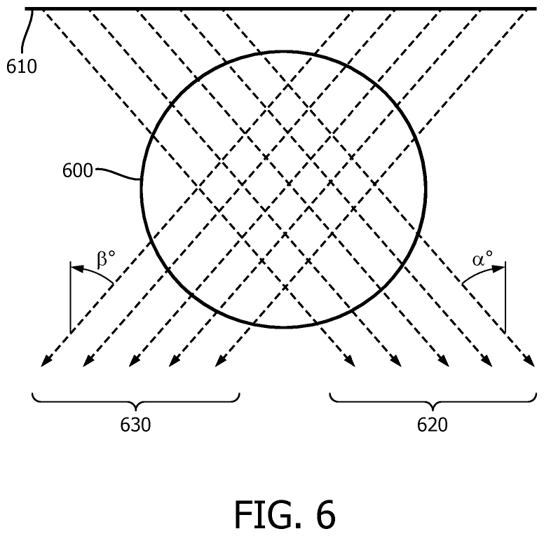

FIG. 6 is a schematic, diagrammatic representation of a circular object 600 being imaged at two different scan angles, in accordance with at least one aspect of the present disclosure. The circular object 600 can be representative of an anatomy inside the body of the patient (e.g., an organ or other anatomy, that is surrounded, adjacent to, or otherwise proximate to different tissue). The anatomy can be referred to as internal anatomy. The inside of the circular object 600 can be representative of one anatomy/tissue type. The area outside of the circular object 600 can be representative of another anatomy/tissue type. The circular object 600 includes a circle-shaped edge (e.g., border, boundary, perimeter, circumference), which shown in FIG. 6. For example, the circle-shaped edge represents the transition between one type of anatomy or tissue and another type of anatomy or tissue. In the example shown in FIG. 6, an ultrasound probe or transducer array 610 emits a first group of ultrasound waves 620 at a beam angle of «° that can be used to generate a first ultrasound subframe of the circular object 600, and a second group of ultrasound waves 630 at a beam angle of β° that can be used to generate a second ultrasound subframe of the object 600. These different angles may for example be achieved through beam steering or through physical reorientation of the ultrasound probe or transducer array 610. Existing spatial compounding algorithms perform simple averaging of the two angled subframes.

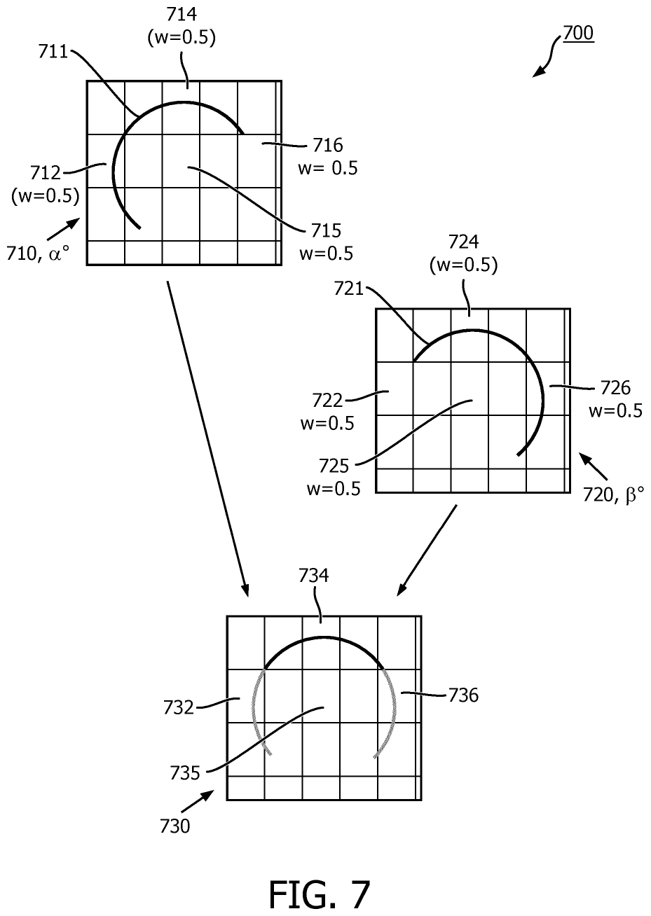

FIG. 7 is a simplified example of a spatial compounding process 700, in accordance with at least one aspect of the present disclosure. A first subframe image 710, captured at an angle α°, includes a first view of the circular object 600 shown in FIG. 6. The first subframe image 710 illustrates a portion 711 of the edge of the circular object 600 in “white”. The portion 711 of the edge of the circular object 600 is illustrated in the first subframe 710 because it is nearer to the source of the ultrasound energy corresponding to the beam angle α° (e.g., the first group of ultrasound waves 620 from the transducer array 610 in FIG. 6), which results in better reflection of the ultrasound energy from the portion 711 of the edge. The opposite portion of the edge of the circular object 600 is not illustrated in the first subframe image 710 because it is farther from the source of the ultrasound energy corresponding to the beam angle α°, which results in poorer reflection of the ultrasound energy from the opposite portion of the edge. For example, the portion 711 corresponds to the portion of the edge of the circular object 600 that is on the near side (relative to the first group of ultrasound waves 620 from the transducer array 610 in FIG. 6), and the opposite portion of the edge of the circular object 600 corresponds the portion of the edge of the circular object 600 that is on the far side (relative to the first group of ultrasound waves 620 from the transducer array 610 in FIG. 6).

The first subframe image 710 includes a pixel 712 that represents a reflection from the portion 711 of the edge of the circular object 600 and is therefore “white” in the image. The first subframe image 710 also includes a pixel 714 that represents a reflection from the portion 711 of the edge of the circular object 600 and is “white” in the image. A pixel 716 does not represent a reflection of the edge of the circular object 600 and is “black” in the image 710. The first subframe image 710 also includes a pixel 715 that is inside of the circular object 600 (and thus does not represent the edge of the circular object 600) and is completely black.

A second subframe image 720, captured at an angle β°, includes a second view of the circular object 600 shown in FIG. 6. The second subframe image 720 illustrates a portion 721 of the edge of the circular object 600 in “white” (different portion than the portion 711 of the edge illustrated in the first subframe image 710). The portion 721 of the edge of the circular object 600 is illustrated in the second subframe 720 because it is nearer to the source of the ultrasound energy corresponding to the beam angle β° (e.g., the second group of ultrasound waves 630 from the transducer array 610 in FIG. 6), which results in better reflection of the ultrasound energy from the portion 721 of the edge. The opposite portion of the edge of the circular object 600 is not illustrated in the second subframe image 720. The opposite portion of the edge of the circular object 600 is not illustrated in the second subframe image 720 because it is farther from the source of the ultrasound energy corresponding to the beam angle β°, which results in poorer reflection of the ultrasound energy from the opposite portion of the edge. For example, the portion 721 corresponds to the portion of the edge of the circular object 600 that is on the near side (relative to the second group of ultrasound waves 630 from the transducer array 610 in FIG. 6), and the opposite portion of the edge of the circular object 600 corresponds the portion of the edge of the circular object 600 that is on the far side (relative to the second group of ultrasound waves 630 from the transducer array 610 in FIG. 6).

The second subframe image 720 includes a pixel 722 that does not represent a reflection of the edge of the circular object 600 and is therefore “black” in the image. The second subframe image 720 also includes a pixel 724 that represents a reflection from the portion 721 of the edge of the circular object 600 and is “white” in the image. A pixel 726 represents a reflection from the portion 721 of an edge of the circular object 600 and is “white” in the image 720. The second subframe image 720 also includes a pixel 725 that inside of the circular object 600 (and thus does not represent the edge of the circular object 600) and is completely black.

Existing spatial compounding algorithms may combine subframes 710 and 720 into a spatially compounded image 730 by simple (e.g., non-weighted or unweighted) averaging of their respective pixels, thus giving a weight of 0.5 to each pixel. Thus, in the example shown in FIG. 7, the spatially compounded image 730 includes a pixel 732 that is an average of pixels 712 (white) and 722 (black) and therefore appears as a medium gray. Similarly, a pixel 734 of the spatially compounded image 730 is an average of pixels 714 (white) and 724 (white), and therefore appears white, whereas pixel 736, an average of pixels 716 (black) and 726 (white) also appears medium gray, and pixel 735, which is an average of pixels 715 (black) and 725 (black), appears black. Thus, the spatially compounded image 730 shows a greater percentage of the circular object 600 than either subframe 710 or 720 by itself, which provides a clear advantage over standard ultrasound imaging, by displaying image frames that contain more complete information.

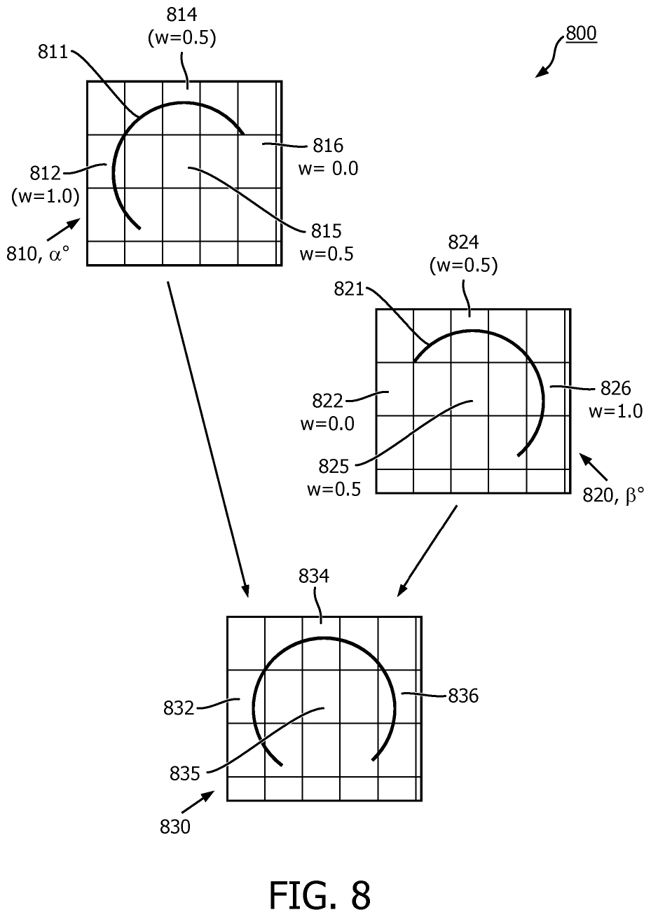

FIG. 8 is a simplified example of an adaptive spatial compounding process 800, in accordance with at least one aspect of the present disclosure. A first subframe image 810, captured at an angle α°, includes a first view of the circular object 600 shown in FIG. 6. As similarly described with respect to FIG. 7, the first subframe image 810 illustrates a portion 811 of the edge of the circular object 600 in “white”. This is because of the portion 811 is closer to the source of the ultrasound energy corresponding to the beam angle c° (e.g., the portion on the near side), which results in better reflection of the ultrasound energy from the portion 811 of the edge. The opposite portion of the edge of the circular object 600 is not illustrated in the first subframe image 810 because it is farther from the source of the ultrasound energy corresponding to the beam angle α° (e.g., the portion on the far side), which results in poorer reflection of the ultrasound energy from the opposite portion of the edge.

The first subframe image 810 includes a pixel 812 that represents a reflection from the portion 811 of the edge of the circular object 600 and is therefore “white” in the image. The first subframe image 810 also includes a pixel 814 that represents a reflection from the portion 811 of the edge of the circular object 600 and is “white” in the image. A pixel 815 does not represent a reflection of the edge of the circular object 600 and is “black” in the image. The first subframe image 810 also includes a pixel 815 that inside of the circular object 600 (and thus does not represent the edge of the circular object 600) and is completely black.

A second subframe image 820, captured at an angle β°, includes a second view of the circular object 600 shown in FIG. 6. As similarly described with respect to FIG. 7, the second subframe image 820 illustrates a portion 821 of the edge of the circular object 600 in “white”. This is because of the portion 821 is closer to the source of the ultrasound energy corresponding to the beam angle β° (e.g., the portion on the near side), which results in better reflection of the ultrasound energy from the portion 821 of the edge. The opposite portion of the edge of the circular object 600 is not illustrated in the second subframe image 820 because it is farther from the source of the ultrasound energy corresponding to the beam angle β° (e.g., the portion on the far side), which results in poorer reflection of the ultrasound energy from the opposite portion of the edge.

The second subframe image 820 includes a pixel 822 that do not represent a reflection from the edge of the circular object 600 and is therefore “black” in the image. The second subframe image 820 also includes a pixel 824 that includes a reflection from the portion 821 of the edge of the circular object 600 and is “white” in the image. A pixel 826 includes a reflection from the portion 821 of the edge of the circular object 600 and is “white” in the image. The second subframe image 820 also includes a pixel 825 that inside of the circular object 600 (and thus does not represent the edge of the circular object 600) and is completely black.

However, unlike the example shown in FIG. 7, the adaptive spatial compounding process 800 of FIG. 8 uses edge detection to compute weights for the pixels of each subframe 810 and 820, so that the spatially compounded image 830 can be produced by a weighted average that gives more relevance to pixels representing an edge or tissue boundary and less relevance to pixels not representing an edge or tissue boundary. In general, an edge can be referred to as a border, a boundary, a perimeter (e.g., an outer or outermost perimeter). Thus, in the example shown in FIG. 8, pixel 812 (white) is given a weight of 1.0 because it represents, or forms a portion of, the edge 811), whereas pixel 822 (black) is given a weight of 0.0 because it does not represent, or form a portion of, the edge 821. Thus, the weighted average pixel 832 of the spatially compounded image 830 appears white.

Similarly, pixels 814 (white) and 824 (white) represent, or form portions of, edges 811 and 821, respectively, and so are given weights of 1.0 which are then normalized to 0.5 each, so that their weights sum to 1.0. Pixel 834, the weighted average of pixels 814 and 824, is therefore white. Pixel 816 (black) does not represent the edge 811, and is given a weight of 0.0, while pixel 826 (white) represents (or forms a portion of) the edge 821, and is given a weight of 1.0, such that pixel 836, the weighted average of 816 and 826, appears white. However, since pixels 815 and 825 are both black and thus do not represent an edge, neither is given extra weighting, and so pixel 835 is a simple average of the two (e.g., a weight of 0.5 for each), and appears black.

Although pixel 815 and pixel 816 are both black, pixel 816 is given a weight of 0.0 because pixel 826 (e.g., the same pixel in an image captured at a different angle) represents the edge 821, whereas pixel 815 is given a weight of 0.5, because pixel 825 (e.g., the same pixel in an image captured at a different angle) does not contain an edge. In general, the weighting depends on presence, absence, or degree of presence/absence of an edge in the same pixel across all of the subframes. The same pixel (corresponding to pixel 816) in at least one of the subframes 810, 820 represents an edge. As a result, that same pixel from the subframe that represents the edge is given the higher weight (pixel 826 from subframe 820 is given weight 1.0), while same pixel from the subject that does not represents the edge is given a lower weight (pixel 816 from subframe 810 is given weight 0.0). The same applies to pixels 812 and 822, and to pixels 816 and 826. Since each of these pixels represents (or forms a portion of) an edge in one subframe and does not represent (or form a portion of) an edge in the other subframe, the pixel representing the edge is given the higher weight (e.g., 1.0), and the pixel that does not represent an edge is given the lower weight (e.g., 0.0).

However, in the case of pixel 815, the same pixel (corresponding to pixel 815) does not represent an edge in any of the subframes, and thus the pixel is given the same weight in all of the subframes. That is, pixel 815 and pixel 825 are the same pixel in different subframes 810, 820. Because neither pixel 815 nor pixel 825 represent an edge, pixel 815 and pixel 825 are given the same weight (0.5).

FIG. 6, FIG. 7, and FIG. 8 are simplified examples. In that regard, the presence or absence of an edge represented in the pixels of FIGS. 7 and 8 has been described in a binary manner (yes the pixel represents an edge vs. no the pixel does not represent an edge). As described below, edge detection can be performed using a continuous scale (instead of binary) such that a pixel in an edge map is associated with a value on the continuous scale representing an extent or degree to which an edge is present/absent (e.g., how sharp is the difference is between two different types of tissue, which represents the edge of an anatomy; how clear is the transition between two types of tissue/anatomy depicted in the ultrasound image frame). Thus, the weighting of a pixel in a given subframe can depend on the degree of presence/absence of an edge represented in the same pixel across all of the subframes.

Thus, the spatially compounded image 830 shows a greater percentage of the circular object 600 than either subframe 810 or 820 by itself, while also providing much greater image contrast (e.g., white pixels instead of gray pixels) than the spatially compounded image 730 of FIG. 7, for image details that show up in only one subframe. Thus, adaptively weighted spatial compounding, where the weights are based on edge detection, provides a clear advantage over existing spatial compounding methods, by displaying image frames that contain not only more complete information, but also higher image contrast or conspicuity for anatomical edges, borders, and partially obscured features.

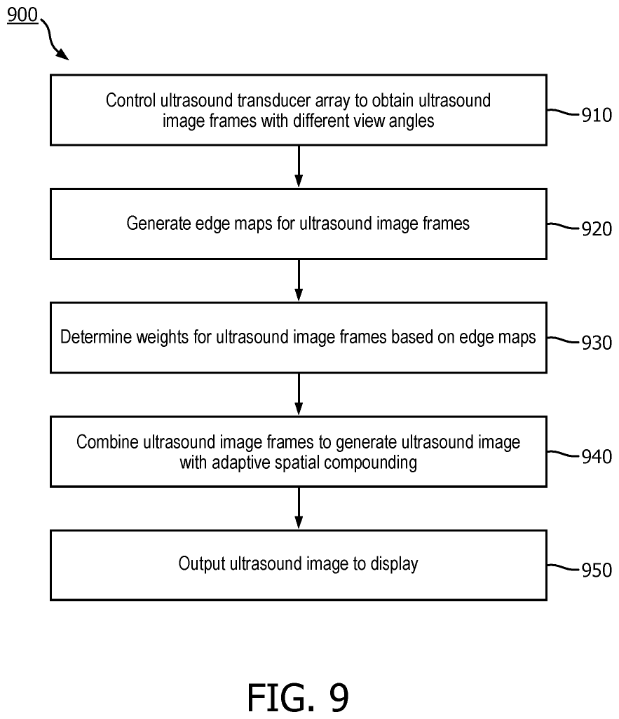

FIG. 9 shows a flow diagram of an example adaptive spatial compounding method 900, in accordance with at least one aspect of the present disclosure. It is understood that the steps of method 900 may be performed in a different order than shown in FIG. 9, additional steps can be provided before, during, and after the steps, and/or some of the steps described can be replaced or eliminated in other aspects. One or more of steps of the method 900 can be carried by one or more devices and/or systems described herein, such as components of the system 100, processor 116, processor 134, and/or processor circuit 250. The method 900 can be stored as instructions of program code recorded on a non-transitory computer-readable storage medium. The instructions are executable by a processor of an ultrasound imaging system to cause the ultrasound imaging system to perform the operations described herein.

In step 910, the method 900 includes controlling the ultrasound image probe's ultrasound transducer array to capture or otherwise obtain ultrasound image subframes with at least two different view angles (e.g., between three and five different view angles), as described above. For example, the method 900 can be performed in real time or near real time using ultrasound image subframes obtained in real time or near real time. The same processor circuit can perform step 910 as well as steps 920-950. In other instances, the method 900 is not performed in real time or near real time. For example, the step 910 can be separated/spaced in time from the steps 920-950. In that regard, the adaptively weighted spatial compounding described herein can be referenced as a post-processing algorithm. In some instances, the step 910 is performed by a first processor circuit in communication with an ultrasound transducer array and/or an ultrasound imaging probe. The steps 920-950 can be performed by a second processor circuit. The first processor circuit and the second processor circuit can be the same or different (e.g., the same ultrasound console or different ultrasound consoles). The method 900 can include the second processor circuit receiving the ultrasound image subframes with at least two different view angles (e.g., between three and five different view angles) that were previously obtained by the ultrasound transducer array. For example, the ultrasound image subframes previously obtained by the ultrasound transducer array can be stored in a memory in direct or indirect communication with the processor (e.g., the memory 138 in FIG. 1, memory 264 in FIG. 2, a memory of a different system 100, a memory of a different processor circuit 250, etc.). Receiving the ultrasound image subframes can include retrieving and/or receiving the stored ultrasound image subframes from the memory. In some instances, the processor circuit can receive the ultrasound image subframes from the ultrasound transducer array. For example, receiving the ultrasound image subframes can include controlling the ultrasound image probe's ultrasound transducer array to capture or otherwise obtain ultrasound image subframes.

In step 920, the method 900 includes generating an edge map for each of the ultrasound image subframes, as described below.

In step 930, the method 900 includes determining weights for each pixel of each ultrasound image subframe based on the pixels of its respective edge map.

In step 940, the method 900 includes combining the ultrasound image subframes (e.g., with a per-pixel weighted average, based on the per-pixel weights determined in step 930) to generate an ultrasound image with adaptive spatial compounding.

In step 950, the method 900 includes outputting the adaptive spatially compounded ultrasound image to a display.

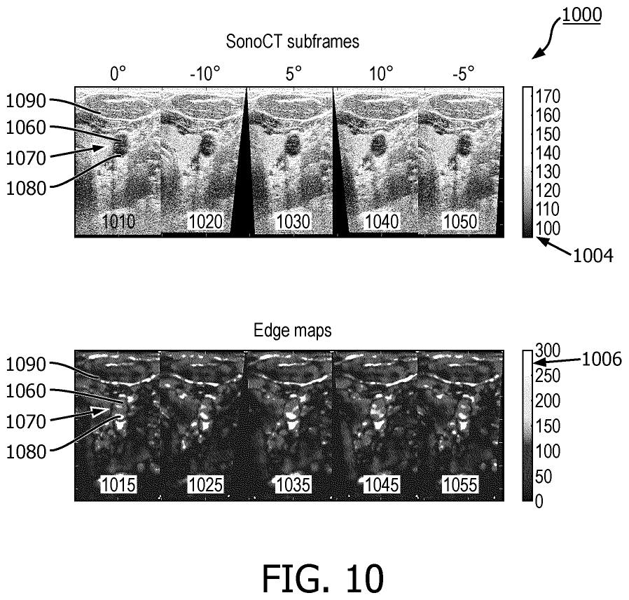

FIG. 10 is a simplified example of an adaptive spatial compounding process 1000, in accordance with at least one aspect of the present disclosure. The adaptive spatial compounding process 1000 improves image contrast and tissue boundary conspicuity by setting increased weights in pixels of the particular subframe that contains the most prominent edge at that pixel location. The subframes are each captured at slightly different incidence angles (e.g., through beam steering, or as a clinician slightly moves or rotates an ultrasound probe in contact with a patient). These subframes can be positionally and rotationally registered to one another in a scan conversion step. For each spatial compounding subframe, a corresponding edge map is produced that highlights transitions in feature brightness (e.g., tissue borders or, more generally, “edges”).

In the example shown in FIG. 10, five different ultrasound subframes 1010, 1020, 1030, 1040, and 1050 (in this case, transverse views of the carotid artery) are captured by the ultrasound probe at five different respective angles 0°, −10°, 5°, 10°, and −5°, as described above in step 910 of FIG. 9. Then, based on the subframes, the adaptive spatial compounding system computes respective edge maps 1015, 1025, 1035, 1045, and 1055 from the ultrasound subframes 1010, 1020, 1030, 1040, and 1050. Example methods for creating the edge map include, but are not limited to, a Frangi filter, a Sobel operator with proper scale matching, or other related edge processing filters.

As observed in the edge maps 1015, 1025, 1035, 1045, and 1055, the edge-finding algorithm retains contrast-generating structures in the image such as the lumen 1060 of the carotid artery 1070 and some prominent segments of the surrounding arterial wall 1080, as well as anatomical borders such as the muscle fascia 1090. Darker colors in the edge maps correspond to pixels that are less likely to represent an edge and lighter colors in the edge maps correspond to pixels that are more likely to represent an edge. For example, the lumen 1060 appears relatively darker in the edge maps because it is the region inside the carotid artery 1070 and thus is unlikely to have an edge.

It is noted that the scale 1004 of the ultrasound subframes 1010, 1020, 1030, and 1040 represents the relative amplitude of the received echoes in decibels (dB), within a range from 100 to 170, whereas the scale 1006 of the edge maps 1015, 1025, 1035, 1045, and 1055, representing the same quantity or a nondimensional edge score, is represented within a range from 0 to 300. This reflects the edge-detection algorithm's scoring of each pixel of the corresponding subframe according to whether there is a prominent edge (higher score) or a non-edge (lower score) at that particular location of the image.

These edge maps 1015, 1025, 1035, 1045, and 1055 can then be used directly as per-pixel weights for the weighted averaging of the subframes 1010, 1020, 1030, 1040, and 1050, thus generating a weight value wₖ for each pixel in a computationally efficient way, such that the subframe with the higher contrast edge is promoted without introducing imaging artifacts. For example, the larger the value of a pixel on the scale 1006, the larger the weight value wₖ for that pixel. Similarly, the smaller the value of a pixel on the scale 1006, the smaller the weight value wₖ for that pixel. Note that in homogeneous areas (e.g., areas without discernible edges), wₖ should revert to roughly equal weights for each subframe, producing the classic spatial compounding image appearance.

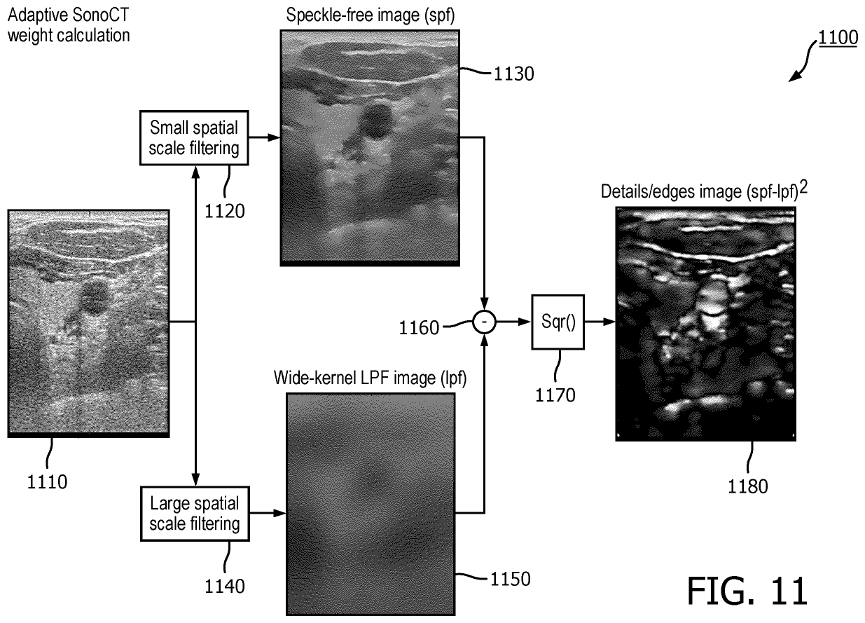

FIG. 11 shows an example adaptive spatial compounding weight calculation method 1100, in a hybrid flow diagram/block diagram form, in accordance with at least one aspect of the present disclosure. The method 1100 may for example be similar or identical to the method 900 of FIG. 9. In one aspect of the method 1100, adaptive spatial compounding processing is applied to the angled subframes acquired with the Philips L12-3 ultrasound system probe using the Vascular Carotid or the Venous LE tissue setting presets (TSPs), as shown above in FIG. 10. The adaptive spatial compounding averaging weights are computed as shown below. In the example shown in FIG. 11, a subframe 1110 is processed to produce an edge map 1180, as described above in step 920 of FIG. 9.

The subframe 1110 is processed with a small spatial scale low pass filtering step or similarly with a high-cutoff frequency low pass filtering step 1120 (e.g., a despeckling algorithm) that eliminates discontinuities from the image that fall below a certain threshold size, resulting in a speckle-free image (spf) 1130. In general, low pass filtering step 1120 can be performed with any linear or nonlinear filter, or any combination of both that suppresses the speckle texture of 1110 while retaining unaffected the tissues, structures and anatomies contained therein. The speckle free image 1130 may for example be estimated by a first step of small kernel median filtering followed by an edge preserving filtering step such as a bilateral filter utilizing Gaussian kernels in both spatial (axial, lateral) and image intensity dimensions. The use of an edge preserving filter may for example ensure that speckle is smoothed out with minimal blurring on the tissue borders or contours and structure edges that need to be enhanced in the final image. Other filters may be used with similar performance, such as the Kuwahara filter, the Lee filter, Guided filters, Nonlocal mean, Anisotropic diffusion filters, and others, whether presently known in the art of hereinafter developed. In other aspects, standard finite impulse response (FIR) and/or infinite impulse response (IIR) filters may be used instead or in addition. However, these need to be appropriately designed to only filter out speckle texture without blurring tissue edges to an unacceptable degree.

The same subframe 1110 is also processed in a low cut-off frequency low-pass filtering or large spatial scale filtering step 1140, to produce a wide-kernel low-pass-filtered image 1150. The wide kernel low-pass-filtered image 1150 (Wide Kernel LPF(Iₖ)) may for example be estimated by 2-D convolution of the image with large kernels in the axial and lateral dimensions. It should be noted that the exact size of the kernels may vary with respect to the image's speckle size and desired feature scale. As an example, in the case of the L12-3 vascular datasets shown in FIG. 10, Hann windows were used with kernel sizes approximately ¼ to ⅓ the size of the image in the axial and lateral dimensions, although other values may be used instead or in addition, and may be tailored to the particular anatomy being imaged.

One exemplary aspect uses a computationally simple multiscale approach to estimate the edge maps of each spatial compounding subframe that can be described by the following relationship:

$$Edge Map ( {I}_{k} ) = {( Speckle Free ( {I}_{k} ) - Wide Kernel LPF ( {I}_{k} ) )}^{2} \qquad ( EQN 1 )$$

In the example shown in FIG. 11, the subtraction of Eqn. 1 is performed at step 1160 to produce a subtraction image (the wide-kernel features are subtracted from the speckle-free image), and the squaring step of Eqn. 1 is performed at step 1170, resulting in the edge map 1180, which has the same dimensions and resolution as the original subframe 1110. In some aspects, an absolute value may be used instead of a squaring function. This formula essentially represents a bandpass filtering operation that excludes large scale brightness variations as well small scale speckle patterns in the image (Wide Kernel LPF (Iₖ) and Speckle (Iₖ)), thus only preserving medium scale tissue edges, bordering areas of structures with different echogenicity and contrast-generating areas such as vessel lumens and cysts. Thus, substituting the subtraction image for the original image 1110, and finding edges in the subtraction image rather than the original image, can yield results that are visually more informative. In other aspects, as described above in FIG. 10, edge maps may be directly generated using more straightforward edge detection algorithms such as the Frangi filter or the Sobel operator with proper scale matching.

The edge map 1180 can then be used as a weight map for a weighted average with other subframes, as described above in steps 930 and 940 of FIG. 9. In some aspects, the edge map 1180 can be used directly. However, in other aspects, the generated edge maps for each image in the spatial compounding ensemble are passed through a sigmoid function to achieve value scaling and/or thresholding, and are subsequently normalized to preserve unit gain in the adaptive spatial compounding weighted summation:

$${w}_{k} ( x , z ) = \frac{g ( Edge Map ( {I}_{k} ) )}{{\sum }_{i = 1}^{K} g ( Edge Map ( {I}_{i} ) ) + eps} , {\sum }_{k = 1}^{K} {w}_{k} ( x , z ) = 1 \qquad ( EQN . 2 )$$

where epsilon normalization is added to avoid division by zero. The obtained weights wₖ are then used to produce the weighted average ASCT (x,z) of the subframes Iₖ. Note that the squaring operation (i.e. Sqr( ) 1170) and the sigmoid/thresholding operation (i.e. g( ) EQN, 2) applied to the difference of the speckle-free image and the wide kernel low-pass filtered image may also be applied in reverse order than the one implied above. For instance, an alternative implementation may be:

$$Edge {Map}^{′} ( {I}_{k} ) = {( g ( Speckle Free ( {I}_{k} ) - Wide Kernel LPF ( {I}_{k} ) ) )}^{2} \qquad ( EQN 3 )$$

$${w}_{k}^{′} ( x , z ) = \frac{Edge {Map}^{′} ( {l}_{k} )}{{\sum }_{i = 1}^{K} Edge {Map}^{′} ( {I}_{k} ) + eps} , {\sum }_{k = 1}^{K} {w}_{k} ( x , z ) = 1 \qquad ( EQN . 4 )$$

This alternative implementation would allow flexibility to enhance differently rising or falling tissue edges. Overall, sigmoid/thresholding operators may be applied both before and after the squaring operation as many times as necessary.

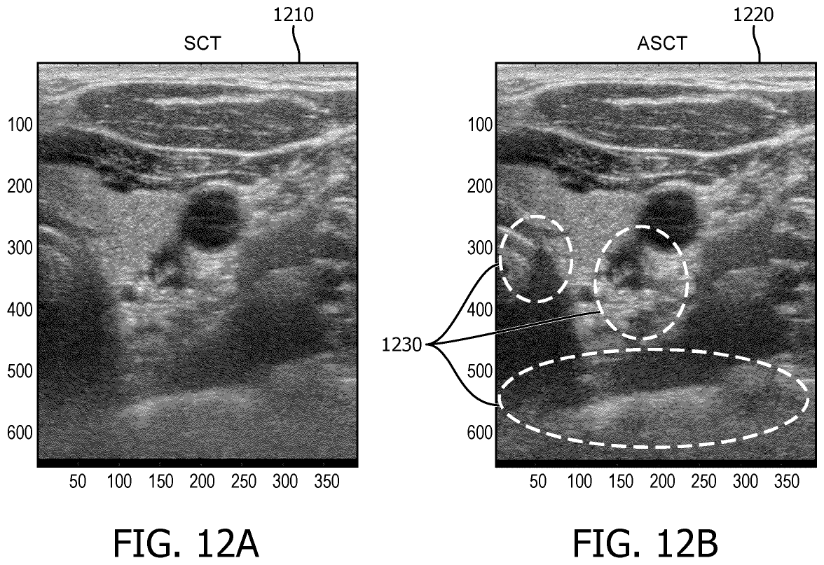

Once the weight maps are available for each subframe that is to be combined, the adaptive spatial compounding system can an adaptive spatial compounding image (e.g., spatially compounded image 1220 of FIG. 12, below), via a weighted average that favors the spatial compounding subframe with the most prominent edge at each pixel. This can produce an image with improved contrast and border delineation. In one aspect the adaptive spatial compounding image is produced with a sum of scalar dot products as follows:

$$ASCT ( x , z ) = {\sum }_{k = 1}^{K} {w}_{k} ( x , z ) \cdot {I}_{k} ( x , z ) \qquad ( EQN . 5 )$$

where ASCT is the adaptive spatial compounding image, Iₖ is the spatial compounding sub-frame corresponding to the kₜₕ steering angle and wₖ is the computed weight. Equation 5 indicates element-wise multiplication of the matrices wₖ, Iₖ, kϵ[1,K] and then summation over k at each pixel location (x,z). In an alternative formulation of Equation 5 in matrix notation, this operation would translate to a Hadamard product followed by addition of the resulting matrices over kϵ[1,K]:

$$\overline{ASCT} = {\sum }_{k = 1}^{K} {\overline{w}}_{k} ⊙ {\overline{I}}_{k} \qquad ( EQN . 6 )$$

where ASCT is the adaptive spatial compounding image, Iₖ is the spatial compounding sub-frame corresponding to the kₜₕ steering angle and wₖ is the computed weight (matrices denoted by the flat accent on top of the variable names).

FIG. 12A shows an example spatial compounding image 1210 produced by existing spatial compounding techniques from the subframes 1010, 1020, 1030, 1040, and 1050 of FIG. 10, in accordance with at least one aspect of the present disclosure. A comparison with the subframes of FIG. 10 will show that the spatially compounded image 1210 has less speckle noise, less shadowing, and generally better delineation of tissue structures. Thus, spatial compounding produces a clear improvement in image quality over the individual subframes.

FIG. 12B shows an example adaptive spatial compounding image 1220 produced by the adaptive spatial compounding system, from the subframes 1010, 1020, 1030, 1040, and 1050 of FIG. 10, in accordance with at least one aspect of the present disclosure. As compared with the standard spatially compounded image 1210 of FIG. 12A, the adaptive spatial compounding image 1220 shows a number of enhanced areas 1230, where tissue boundaries and other related structures are more sharply defined. In particular, the adaptive spatial compounding image 1220 tissue shows structures more clearly at greater imaging depths (e.g., toward the bottom of the image). Thus, adaptive spatial compounding, using weights derived from edge maps, produces a clear improvement in image quality over existing spatial compounding techniques.

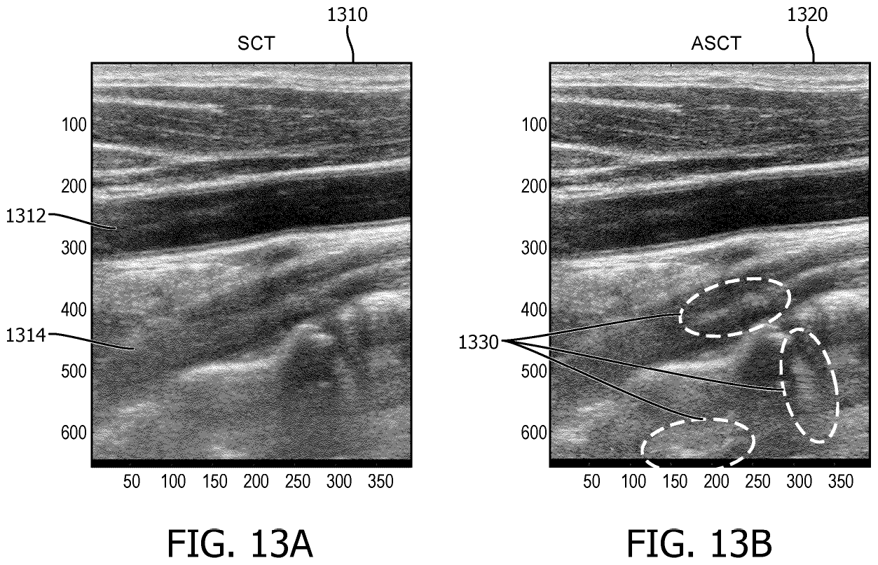

FIG. 13A shows an example spatial compounding image 1310 produced by existing spatial compounding techniques from subframes 410, 420, 430, 440, and 450 of FIG. 4, in accordance with at least one aspect of the present disclosure. The image shows a lateral view of a blood vessel 1312 and surrounding tissue 1314. A comparison with the subframes of FIG. 4 will show that the spatially compounded image 1310 has less speckle noise, less shadowing, and generally better delineation of tissue structures. Thus, spatial compounding produces a clear improvement in image quality over the individual subframes.

FIG. 13B shows an example adaptive spatial compounding image 1320 produced by the adaptive spatial compounding system, in accordance with at least one aspect of the present disclosure. As compared with the standard spatially compounded image 1310 of FIG. 13A, the adaptive spatial compounding image 1320 shows a number of enhanced areas 1330, where tissue boundaries and other related structures are more sharply defined. In particular, the adaptive spatial compounding image 1320 tissue shows structures more clearly at greater imaging depths (e.g., toward the bottom of the image). Thus, adaptive spatial compounding, using weights derived from edge maps, produces a clear improvement in image quality over existing spatial compounding techniques.

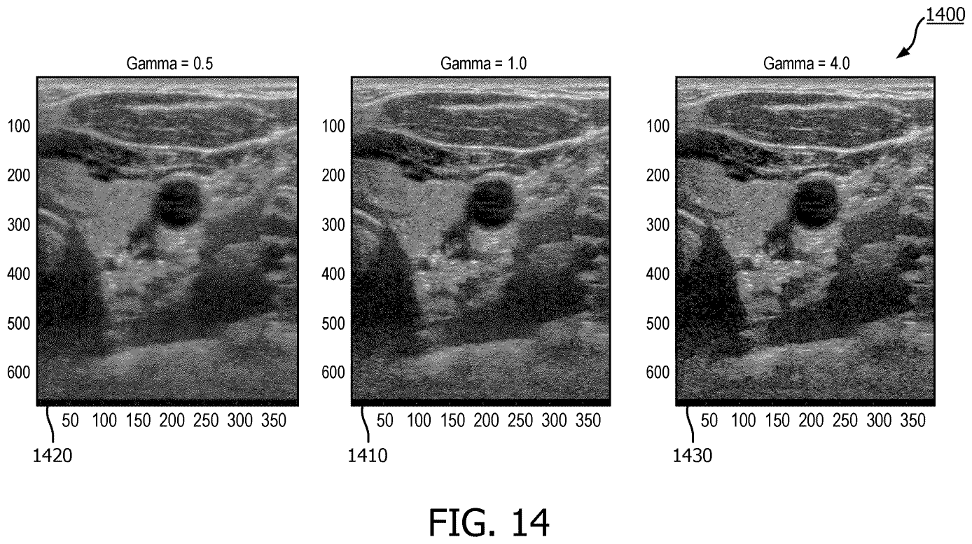

FIG. 14 is an illustration 1400 of the effect of gamma correction on an adaptive spatial compounding system image, in accordance with at least one aspect of the present disclosure. Gamma correction can be a nonlinear (e.g., power-law) operation on the luminance values of an image. A gamma value of 1 leaves the image unchanged, whereas a gamma value of less than 1 compresses the luminance values, e.g., decreases the difference between the minimum and maximum luminance values in the image, and a gamma value of greater than 1 expands the luminance values, e.g., increases the difference between the minimum and maximum luminance values in the image. In ultrasound imaging, tissue borders and transitions may become sharper as gamma increases. For gamma values less than 1, the adaptive spatial compounding system image's appearance may be closer to those produced by standard spatial compounding, whereas the image enhancements created by adaptive spatial compounding based on edge detection may be more apparent for gamma values greater than 1.

In some aspects, gamma correction may be applied to the adaptive spatial compounding image itself. In other aspects, instead or in addition, gamma correction may be applied to the edge maps prior to normalization to modulate the aggressiveness of the algorithm, as shown in the following relationship:

$${w}_{k , gamma} ( x , z ) = \frac{{g ( Edge Map ( {I}_{k} ) )}^{gamma}}{{\sum }_{i = 1}^{K} {g ( Edge Map ( {I}_{i} ) )}^{gamma} + eps} , \qquad ( Eqn . 7 )$$

$${\sum }_{k = 1}^{K} {w}_{k , gamma} ( x , z ) = 1$$

Increasing gamma past 1 tends to stretch edge map intensities, further favoring one of the spatial compounding subframes over the others while decreasing gamma below 1 tends to compress edge map intensities, bringing the performance of adaptive spatial compounding closer to that of default spatial compounding. Examples illustrating such gamma correction effects are shown in FIG. 14. An alternative aspect to diminish the aggressiveness of the algorithm would be to perform spatial smoothing of the edge maps prior to using them in the weighted averaging step. Note that any operations on the edge maps may be applied in different order and more times than what is implied by the above formula. For example, gamma correction applied first, then sigmoid/thresholding etc.

In the example shown in FIG. 14, a gamma=1.0 image 1410 is similar in appearance to the adaptive spatial compounding image 1220 of FIG. 12B, whereas a gamma=0.5 image 1420 is similar in appearance to the standard spatial compounding image 1210 of FIG. 12A. However, a gamma=4.0 image 1430 provides generally sharper definition of tissue structures, and may therefore be desirable for certain applications. It is noted that excessively high gamma values can create a grainy, starkly black-and-white appearance to ultrasound images, and may therefore decrease rather than increase the quality of the image. For this reason, “standard” or “preset” gamma values may be application-specific, e.g., a particular gamma value may produce clear images of the vasculature of the kidney, whereas a different gamma value may produce clear images of the lungs. In some aspects, gamma may be a real-time user-selectable value, e.g., with a knob or slider.

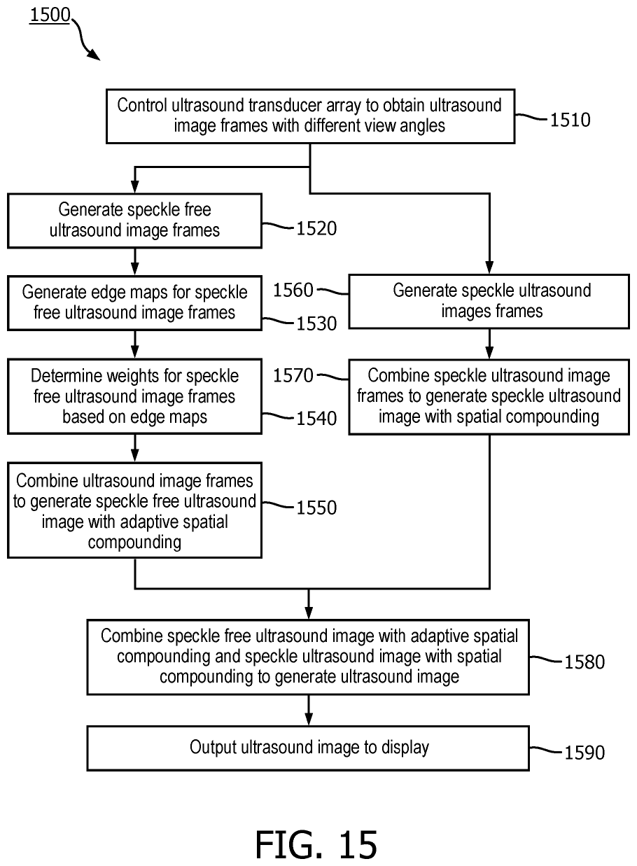

FIG. 15 shows a flow diagram of an example 2-scale adaptive spatial compounding method 1500, in accordance with at least one aspect of the present disclosure. Adaptive spatial compounding can, in some cases, increase the speckle size of an image as a side effect of the image processing. While adaptive spatial compounding improves tissue border conspicuity, the weighted averaging approach may not be optimal for the suppression of speckle variance. To address this issue, the aspect shown in FIG. 15 uses a 2-scale process to combine the improved contrast and tissue appearance of adaptive spatial compounding with the widely accepted appearance of speckling in non-adaptively weighted spatial compounding images. This process involves first performing adaptive spatial compounding on the speckle-free components of the subframes as computed by an edge-preserving filtering operation (Bilateral, Lee filter etc.) or a conventional small kernel low pass filtering operation, and then performing non-adaptively weighted spatial compounding on the speckle components of the subframes as computed by subtracting the speckle-free component from the original image.

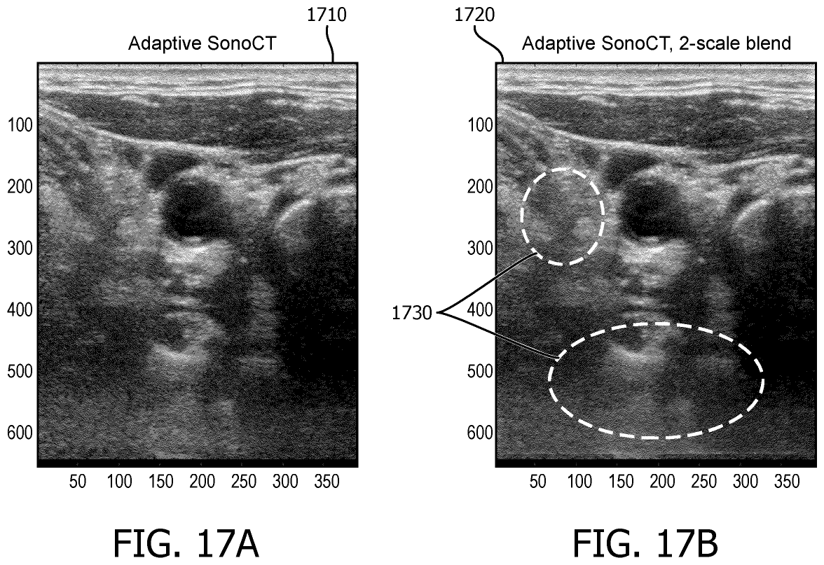

It should be noted that the spatial scale of the filtering operation to estimate the speckle-free component of spatial compounding subframes plays a role in the aggressiveness of the technique and its effects on image quality. The wider the spatial kernels used in the filtering, the less the tissue border conspicuity and contrast improvements are and the more the image looks like an non-adaptively weighted spatial compounding image. Examples comparing non-adaptively weighted, adaptive and 2-scale adaptive spatial compounding are shown in FIG. 17.

It is understood that the steps of method 1500 may be performed in a different order than shown in FIG. 15, additional steps can be provided before, during, and after the steps, and/or some of the steps described can be replaced or eliminated in other aspects. One or more of steps of the method 1500 can be carried by one or more devices and/or systems described herein, such as components of the system 100, processor 116, processor 134, and/or processor circuit 250. The method 1500 can be stored as instructions of program code recorded on a non-transitory computer-readable storage medium. The instructions are executable by a processor of an ultrasound imaging system to cause the ultrasound imaging system to perform the operations described herein.

In step 1510, the method 1500 includes controlling the ultrasound transducer array to obtain ultrasound image subframes with at least two different view angles (e.g., between three and five different view angles), as described above. As similarly described above with respect to the method 900 (FIG. 9), the method 1500 can be performed in real time or near real time, or the method 900 is not performed in real time or near real time. For example, the method 1500 can include receiving previously obtained ultrasound image subframes, such that the adaptively weighted spatial compounding described herein can be referenced as a post-processing algorithm.

In step 1520, the method 1500 includes generating speckle-free image subframes from the obtained subframes, e.g., by running a high-pass filter or despeckle algorithm on the images.

In step 1530, the method 1500 includes generating an edge map for each of the despeckled ultrasound image subframes, as described below.

In step 1540, the method 1500 includes determining weights for each pixel of each despeckled ultrasound image subframe based on the pixels of its respective edge map.

In step 1550, the method 1500 includes combining the despeckled ultrasound image subframes (e.g., with a per-pixel weighted average, based on the per-pixel weights determined in step 1540) to generate an ultrasound image with adaptive spatial compounding.

In step 1560, the method 1500 includes generating speckle image subframes from the obtained ultrasound image subframes. This can be done for example by setting each pixel value to the subtraction of the despeckled image subframes from the obtained (raw) ultrasound subframes.

In step 1570, the method 1500 includes combining the speckle image subframes to generate a speckle ultrasound imaging with non-adaptively weighted (e.g., non-weighted, unweighted, or equally weighted) spatial compounding, for example with unweighted average or an linear or nonlinear unweighted function other than averaging.

In step 1580, the method 1500 includes combining the despeckled, adaptive spatially compounded ultrasound image with the non-adaptively weighted spatially compounded speckle image to generate a final, 2-scale adaptive spatially compounded ultrasound image. This may be done for example by simply adding the images. Furthermore, if needed rescaling may be employed to adjust the average pixel brightness. For display purposes, the combined spatially compounded image can then be substituted for the speckle-free spatially compounded image, thus providing a more informative display to the user.

In step 1590, the method 900 includes outputting the 2-scale adaptive spatially compounded ultrasound image to a display.

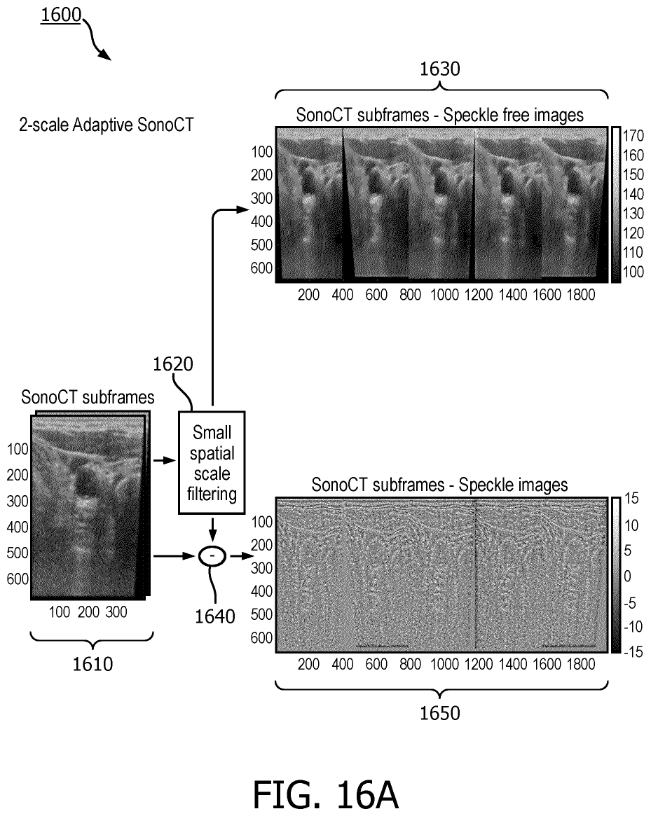

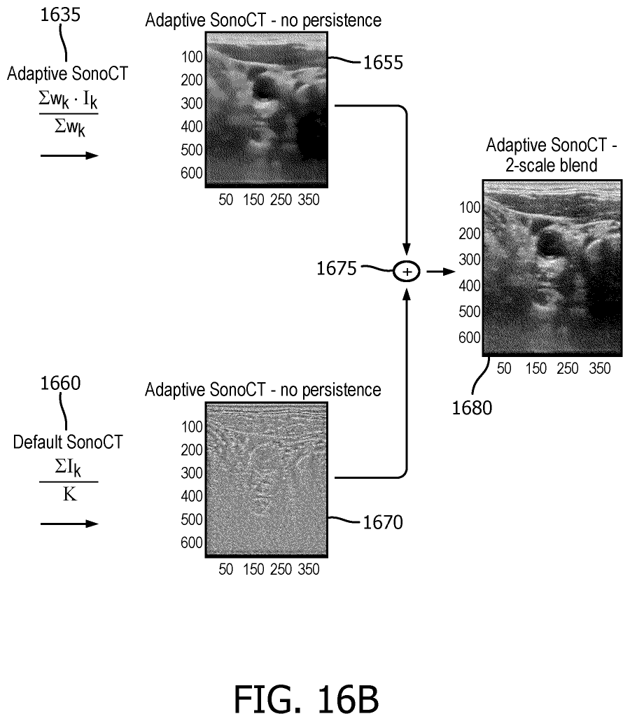

FIG. 16 shows an example 2-scale adaptive spatial compounding weight calculation method 1600, in a hybrid flow diagram/block diagram form, in accordance with at least one aspect of the present disclosure. The method 1600 may for example be similar or identical to the method 1500 of FIG. 15. In one aspect of the method 1600, adaptive spatial compounding processing is applied to the angled subframes acquired with the L12-3 probe using the Vascular Carotid or the Venous LE TSPs, as shown above in FIG. 10. In the example shown in FIG. 16, a group of subframes 1610 is processed to produce a 2-scale spatially compounded image 1680, as described above in FIG. 15.

The subframes 1610 are processed through a small-scale filtering step 1620 to produce despeckled images 1630, which are fed into an adaptive spatial compounding step 1635 as described above, to produce an adaptive spatially compounded image 1655. The same subframes 1610 are processed through a subtraction step 1640 to produce speckle images 1650. The speckle images 1650 are fed into a standard (e.g., non-weighted, unweighted, or equally weighted) spatial compounding step 1660, to produce a spatially compounded speckle image 1670. In an addition step 1675, the adaptive spatially compounded image 1655 is then combined with the spatially compounded speckle image 1670 to produce the 2-scale spatially compounded image 1680.

FIG. 17A shows an example adaptive spatial compounding image 1710 produced by the adaptive spatial compounding system, from the subframes 1010, 1020, 1030, 1040, and 1050 of FIG. 10, in accordance with at least one aspect of the present disclosure. Image 1710 may for example be comparable to image 1220 of FIG. 12B. As compared with the standard spatially compounded image 1210 of FIG. 12A, the adaptive spatial compounding image 1220 shows a clear improvement in image quality.

FIG. 17B shows an example 2-scale adaptive spatial compounding image 1720 produced by the adaptive 2-scale spatial compounding system of FIGS. 15 and 16, from the subframes 1010, 1020, 1030, 1040, and 1050 of FIG. 10, in accordance with at least one aspect of the present disclosure. As compared with image 1710 of FIG. 17A, image 1720 shows enhanced areas 1730, where speckle is reduced and tissue boundaries are more clearly delineated. Thus, the 2-scale adaptive spatial compounding method shows clear advantages over the adaptive spatial compounding method of FIGS. 9 and 11.

As will be readily appreciated by those having ordinary skill in the art after becoming familiar with the teachings herein, the adaptive spatial compounding system advantageously improves image quality, tissue boundary contrast, and imaging depth of an ultrasound imaging system, without user intervention and without increasing user workload.

It is noted that flow diagrams and block diagrams are provided herein for exemplary purposes; a person of ordinary skill in the art will recognize myriad variations that nonetheless fall within the scope of the present disclosure.

For example, block diagrams may show a particular arrangement of components, modules, services, steps, processes, or layers, resulting in a particular data flow. It is understood that some aspects of the systems disclosed herein may include additional components, that some components shown may be absent from some aspects, and that the arrangement of components may be different than shown, resulting in different data flows while still performing the methods described herein.

Similarly, the logic of flow diagrams may be shown as sequential. However, similar logic could be parallel, massively parallel, object oriented, real-time, event-driven, cellular automaton, or otherwise, while accomplishing the same or similar functions. In order to perform the methods described herein, a processor may divide each of the steps described herein into a plurality of machine instructions, and may execute these instructions at the rate of several hundred, several thousand, several million, or several billion per second, in a single processor or across a plurality of processors. Such rapid execution may be necessary in order to execute the method in real time or near-real time as described herein. For example, in order to compute spatially compounded ultrasound images for a real-time ultrasound video stream, the system may need to be capable of computing edge maps and per-pixel weighted averages for a moving window of 2-5 sub-frames, and combining the sub-frames into a spatially compounded image, at the same frame rate as the ultrasound video stream itself (e.g., 30 Hz, 60 Hz, etc.).

A number of variations are possible on the examples and aspects described above. For example, while most results in the present disclosure are shown for a linear ultrasound probe and for vascular applications, the adaptive spatial compounding system can also be utilized by other imaging systems where spatial compounding is currently employed. However, a different tuning of the weighing scheme may be necessary in those cases to obtain optimal results. For example, modulation of the weighting scheme according to the image brightness or image signal-to-noise ratio (SNR) at each pixel may be necessary to avoid introducing unwanted image effects and artifacts. For instance, adaptive spatial compounding may be useful in OB applications where increased sharpness of the delicate, deep-lying fetal structures is desirable. However, some refinement of the weights may be necessary to avoid deepening acoustic shadows that are relatively common in OB datasets (e.g., baby rib shadows). More generally, the selection of edge-detection algorithms, weights, and gamma values may be tissue-specific, e.g., as part of an imaging preset that includes imaging parameters optimized for particular tissue types.

One issue arising in some imaging scenarios with significant tissue or probe motion is that since the subframes are not well aligned, some distortion of tissue and jitter may be observed in the adaptive spatial compounding images. Non-adaptively weighted spatial compounding also suffers from a similar issue in the presence of motion, but its manifestation is increased image blurriness which may be more acceptable in a clinical setting. However, it should be noted that when replaying results on the imaging system, non-real-time adaptive spatial compounding issues may not be as noticeable, and may possibly be addressed by the imaging system's persistence mechanisms. A strategy that can be implemented to fully rectify this issue involves computing a metric for subframe alignment and, in the case of significant frame misalignment, reverting the adaptive spatial compounding weights back to equal weights. In practice, this approach switches adaptive spatial compounding back to non-adaptively weighted spatial compounding in the presence of significant motion. Motion compensation efforts to align the spatial compounding subframes can also improve adaptive spatial compounding performance in the presence of significant motion. Alternatively, smoothing out the edge maps can also provide improved performance in the presence of motion.

The adaptive spatial compounding system can be implemented as a selectable or non-selectable enhancement system supporting non-adaptively weighted spatial compounding, and can provide a substantial boost in image quality for applications where enhanced tissue edges and border delineation is of high clinical significance. For example, in atherosclerotic carotid imaging, adaptive spatial compounding based on edge detection may aid in better delineating the limits between atherosclerotic plaque and the lumen of the vessel. Additionally, the adaptive spatial compounding system could be very important in clinical scenarios where the accurate and clear delineation of cancerous lesions is of high importance (e.g., thyroid, breast). Additionally, since this modality also conveys the impression of clarity at depth, it may be useful in obstetric (OB) applications.

The logical operations making up the aspects of the technology described herein are referred to variously as operations, steps, objects, elements, components, or modules. It should be understood that these may occur or be performed or arranged in any order, unless explicitly claimed otherwise or a specific order is inherently necessitated by the claim language.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the adaptive spatial compounding system. Connection references, e.g., attached, coupled, connected, joined, or “in communication with” are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the adaptive spatial compounding system as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. For example, even though performance was showcased in 2-D B-mode images, the input data to the adaptive spatial compounding may include data at earlier or later processing stages than the images depicted in the figures herein. The input may include 8-bit or 16-bit B-mode data, RF-signal data (i.e. prior to envelope detection and log compression), or even per-channel RF-signal data. Additionally, this technology may also be applied to 3-D ultrasound volumes acquired with different look directions using multidimensional array transducers. Furthermore, the adaptive spatial compounding algorithm described herein may be used to combine ultrasound images acquired not just with different look directions but also with variations in other acquisition parameters. This way, a combined image with optimal contrast and tissue conspicuity may be obtained from the range of different imaging parameters employed.

It is intended that all matter contained in the above description and shown in the accompanying drawings shall be interpreted as illustrative only of particular aspects and not limiting. Changes in detail or structure may be made without departing from the basic elements of the subject matter as defined in the following claims.

1. An ultrasound imaging system, comprising:
a processor configured for communication with a display, wherein the processor is configured to:
receive a plurality of images of a patient's internal anatomy obtained by an ultrasound imaging probe at a respective plurality of different angles;
for each respective image of the plurality of images, compute an edge map;
using the edge maps as weight maps, compute a weighted function of each corresponding pixel of each image in the plurality of images;
assemble a spatially compounded image based on values of the weighted functions of each corresponding pixel; and
output the spatially compounded image on the display.

2. The ultrasound imaging system of claim 1, further comprising the ultrasound imaging probe.

3. The ultrasound imaging system of claim 2, wherein, to receive the plurality of images, the processor is configured to control the ultrasound imaging probe to obtain the plurality of images at the respective plurality of different angles.

4. The ultrasound imaging system of claim 1,
further comprising a memory in communication with the processor,
wherein, to receive the plurality of images, the processor is configured to retrieve the plurality of images from the memory.

5. The ultrasound imaging system of claim 1, wherein the processor is further configured to, for each respective image of the plurality of images, prior to computing the edge map:
generate a corresponding despeckled image;
generate a corresponding low-pass-filtered image;
generate a corresponding subtraction image by subtracting the corresponding low-pass-filtered image from the corresponding despeckled image; and
substitute the corresponding subtraction image for the respective image.

6. The ultrasound imaging system of claim 1, wherein the processor is further configured to, prior to outputting the spatially compounded image, perform gamma correction on the spatially compounded image.

7. The ultrasound imaging system of claim 1, wherein the processor is further configured to:
for each respective image of the plurality of images:
generate a corresponding despeckled image;
generate a corresponding speckle image by subtracting the respective image from the despeckled image and replacing each pixel value with a corresponding square or absolute value of the pixel value, thus forming a plurality of speckle images;
assemble a spatially compounded speckle image by computing a non-weighted function of each corresponding pixel of the plurality of speckle images; and
add the spatially compounded speckle image to the spatially compounded image.

8. The ultrasound imaging system of claim 1, wherein a pixel representing an edge is enhanced in the spatially compounded image as compared with the plurality of images.

9. The ultrasound imaging system of claim 1, wherein a pixel representing a shadow is de-emphasized in the spatially compounded image as compared with the plurality of images.

10. The ultrasound imaging system of claim 1, wherein a pixel representing a side lobe artifact or grating lobe artifact is de-emphasized in the spatially compounded image as compared with the plurality of images.

11. The ultrasound imaging system of claim 1, wherein, to use the edge maps as weight maps, the processor is configured to:
pass the edge maps through a sigmoid function to achieve value scaling or thresholding; and
normalize pixel values of the edge maps such that summing a given pixel across all of the edge maps yields a value of 1.

12. The ultrasound imaging system of claim 1, wherein the processor is further configured to align the images of the plurality of images with scan conversion.

13. The ultrasound imaging system of claim 1, wherein the weighted function comprises a weighted average.

14. An ultrasound imaging method, comprising:
receiving, with a processor, a plurality of images obtained by an ultrasound imaging probe at a respective plurality of different angles;
computing, with the processor, an edge map for each respective image of the plurality of images;
computing, with the processor, a weighted function of each corresponding pixel of each image in the plurality of images, using the edge maps as weight maps;
assembling, with the processor, a spatially compounded image based on values of the weighted functions of each corresponding pixel; and
outputting, with the processor, the spatially compounded image on a display in communication with the processor.

15. A non-transitory computer-readable storage medium having program code recorded thereon, wherein the program code comprises instructions executable by a processor of an ultrasound imaging system to cause the ultrasound imaging system to:
receive a plurality of images of a patient's internal anatomy obtained by an ultrasound imaging probe at a respective plurality of different angles;
for each respective image of the plurality of images, compute an edge map;
using the edge maps as weight maps, compute a weighted function of each corresponding pixel of each image in the plurality of images;
assemble a spatially compounded image based on values of the weighted functions of each corresponding pixel; and
output the spatially compounded image on a display in communication with the processor.
